(19) United States
(12) Patent Application Publication
(10) Pub. No.: US 2024/0382435 A1
(43) Pub. Date: Nov. 21, 2024

(54) NON-CERULOPLASMIN BOUND COPPER LEVEL MONITORING

(71) Applicant: ORPHALAN S.A., 75011 Paris (FR)

(72) Inventors: Naseem Amin, London Greater London (GB); Timothy James Morley, Harrogate Yorkshire (GB); Omar Kamlin, 75011 Paris (FR)

(21) Appl. No.: 18/288,322

(22) PCT Filed: Apr. 26, 2022
(86) PCT No.: PCT/EP2022/061003

§ 371 Date: Oct. 25, 2023

(30) Foreign Application Priority Data
Apr. 26, 2021 (EP) 21305537.9
Apr. 27, 2021 (EP) 21305544.5

(51) Int. Cl. A61K 31/132; A61K 31/198; A61P 3/12; G01N 30/02; G01N 30/72; G01N 33/84; H01J 49/10

(52) U.S. Cl. CPC A61K 31/132; A61K 31/198; A61P 3/12; G01N 30/7233; G01N 33/84; H01J 49/105; G01N 2030/027; G01N 2800/52

(57) ABSTRACT

The present invention relates to the treatment and/or monitoring of subjects suffering from, or susceptible to, Wilson's Disease, and involves measuring the serum non-ceruloplasmin (NCC) level in a subject and determining whether the serum NCC level is within a specified range. Treatment regimes can be adjusted if appropriate to control the subject's serum NCC level to be within the desired range.

### BACKGROUND OF THE INVENTION

### Field of the Invention

The present invention relates to the treatment and/or monitoring of subjects suffering from, or susceptible to, Wilson's Disease, and involves measuring the serum non-ceruloplasmin (NCC) level in a subject and determining whether the serum NCC level is within a specified range. Treatment regimes can be adjusted if appropriate to control the subject's serum NCC level to be within the desired range.

### Background to the Invention

Wilson's Disease is an autosomal recessive disorder resulting in impairment of copper transport functions and results in accumulation of copper in major organs including liver, brain, eyes and kidney. A mutation in the ATP7B gene, located on chromosome 13, is responsible for Wilson's Disease. This gene codes for the ATP7B protein that mediates the binding of copper molecules, forming ceruloplasmin, to transport the bound copper to its intended sites and into the bile for subsequent biliary excretion.

In Wilson's Disease patients, copper progressively accumulates in the liver and can also be deposited in other tissues and organs such as the brain, kidneys and cornea. Untreated, or with poor adherence to drug therapy, Wilson's Disease is universally fatal. Prognosis for survival depends on the severity of liver and neurological disease, and compliance with drug treatment.

Treatment of Wilson's Disease aims to control copper levels, principally via removal of excess copper from the body. Current treatments involve the administration of a copper (II) chelator such as a trientine (triethylenetetramine) salt, including trientine dihydrochloride and trientine tetrahydrochloride, or D-penicillamine.

For the diagnosis of patients with Wilson's Disease and as an assessment of treatment adequacy, compliance and adherence, patient evaluation includes an ongoing assessment of multiple clinical parameters and the use of in vitro diagnostic tests to assess copper levels. Typically, diagnosis and ongoing patient assessment is performed using a combination of a 24 hour urinary copper excretion (UCE) test, tests for the circulating levels of serum non-ceruloplasmin bound copper (NCC) and total copper concentration, alanine transaminase (ALT) tests, clinical determination of the presence or absence of Kayser-Fleischer rings, analysis of liver function and liver copper levels and clinical analysis of neurological symptoms using the Unified Wilson's Disease Rating Scale. The need for multiple tests to achieve accurate patient monitoring and/or diagnosis makes monitoring and diagnosing Wilson's Disease time-consuming, resource-heavy and can result in decreased patient compliance with testing.

The standard approach to determining NCC uses an indirect EDTA ultra-centrifugation method (El Balkhi and Poupon et al (2009), “Determination of ultrafiltrable and exchangeable copper in plasma: stability and reference values in healthy subjects”, Anal. Biol. Chem., 394(5):1477-1484). In the EDTA method, sera are treated with EDTA to chelate copper not bound to ceruloplasmin and then ultra-filtrated to remove copper bound to ceruloplasmin. However, the inventors have now found that there are multiple deficiencies to this approach, for example it has been found that at certain concentrations EDTA may actually displace copper from ceruloplasmin and may not measure all copper bound to albumin.

There is therefore a need to develop improved assays which can be accurately and reliably used to monitor and/or assess a patient who has, or may have, Wilson's Disease. In particular, there is a need to provide assays which can improve patient care by monitoring patient responsiveness to therapy to allow optimized dosing, and by reducing the number of clinical tests that are required for accurate assessment of the patient.

### SUMMARY OF THE INVENTION

The present inventors have shown that assessment of serum non-ceruloplasmin copper levels using a copper speciation assay provides an accurate method for assessing and monitoring patients who have, or are at risk of having, Wilson's Disease. The copper speciation assay has been found to be more accurate than the standard EDTA method, in which copper from albumin becomes bound to both a high molecular weight artefact (which remains above the filter) and EDTA (which passes through the filter) and accounts for an underestimate of NCC. In particular, the present inventors have shown that assessment of serum non-ceruloplasmin copper levels using a copper speciation assay can be used as an accurate clinical tool without the need for other companion assessments, such as 24 hour UCE. Furthermore, the inventors have determined how Wilson's Disease can be better controlled within a subject, by monitoring the serum non-ceruloplasmin copper levels using a copper speciation assay and adapting the treatment of the subject accordingly. The accuracy of the copper speciation assay allows the serum non-ceruloplasmin copper levels of the patient to be tightly controlled within a specific range that may provide an improved clinical outcome for patients with Wilson's Disease. In particular, by controlling non-ceruloplasmin copper levels in accordance with the invention, patients may maintain stable disease (i.e. levels of liver enzymes such as alanine aminotransferase (ALT) within normal range or <1.5× upper limit of normal (ALT<82)). Such patients may maintain stable disease for the period of their treatment, or over a period of at least 4, 8, 12, 16, 20, 24 or 48 weeks.

The present invention therefore provides a copper (II) chelator for use in a method of treating Wilson's Disease in a subject, wherein the method comprises
measuring a serum non-ceruloplasmin bound copper level of the subject using a copper speciation assay; and
controlling the subject's serum non-ceruloplasmin bound copper level to be in the range of from 25 to 130 ng/mL.

The present invention also provides a copper (II) chelator for use in a method of monitoring a subject undergoing treatment for Wilson's Disease by administration of the copper (II) chelator, wherein the method comprises
measuring a serum non-ceruloplasmin bound copper level of the subject using a copper speciation assay; and
controlling the subject's serum non-ceruloplasmin bound copper level to be in the range of from 25 to 130 ng/mL.

Preferably, the range is from 40 to 80 ng/mL.

Typically, said measuring step is repeated one or more times during the subject's treatment, such the subject's serum non-ceruloplasmin levels are monitored during their treatment.

Preferably, the method comprises measuring or monitoring the subject's serum non-ceruloplasmin bound copper level and, in the case that the subject's serum non-ceruloplasmin bound copper level falls outside the range of from 40 to 80 ng/mL, adjusting the dose of copper (II) chelator, and optionally maintaining the adjusted dose until the subject's serum non-ceruloplasmin bound copper level is once again within the range of from 40 to 80 ng/mL.

Preferably, said adjustment comprises increasing the dose of the copper (II) chelator if the subject's serum non-ceruloplasmin bound copper level falls above 80 ng/mL, preferably increasing the dose by 150 to 300 mg/day of active chelator, or reducing the dose of the copper (II) chelator if the subject's serum non-ceruloplasmin bound copper level falls below 40 ng/mL preferably reducing the dose by 150 to 300 mg/day of active chelator.

Preferably, the copper (II) chelator is trientine tetrahydrochloride or D-penicillamine, more preferably trientine tetrahydrochloride.

The present invention also provides the use of a copper (II) chelator in the manufacture of a medicament for use in the treatment of Wilson's Disease in a subject, wherein the treatment comprises
measuring a serum non-ceruloplasmin bound copper level of the subject using a copper speciation assay; and
controlling the subject's serum non-ceruloplasmin bound copper level to be in the range of from 25 to 130 ng/mL, preferably from 40 to 80 ng/mL,
preferably wherein the copper (II) chelator is trientine tetrahydrochloride.

The present invention also provides the use of a copper (II) chelator in the manufacture of a medicament for use in monitoring a subject undergoing treatment of Wilson's Disease by administration of the copper (II) chelator, wherein the monitoring comprises
measuring a serum non-ceruloplasmin bound copper level of the subject using a copper speciation assay; and
controlling the subject's serum non-ceruloplasmin bound copper level to be in the range of from 25 to 130 ng/mL, preferably from 40 to 80 ng/mL,
preferably wherein the copper (II) chelator is trientine tetrahydrochloride.

The present invention also provides a method of monitoring a subject undergoing treatment for Wilson's Disease, wherein the method comprises
measuring a serum non-ceruloplasmin bound copper level of the subject using a copper speciation assay; and
controlling the subject's serum non-ceruloplasmin bound copper level to be in the range of from 25 to 130 ng/mL, preferably from 40 to 80 ng/mL.

Preferably, the subject is undergoing treatment for Wilson's Disease by administration of a copper (II) chelator. An effective amount of a copper (II) chelator is typically administered, which is generally provided as a plurality of doses of copper (II) chelator.

Preferably, the copper (II) chelator is trientine tetrahydrochloride or D-penicillamine, preferably trientine tetrahydrochloride.

The present invention also provides a method of treating Wilson's Disease in a subject, wherein the method comprises
measuring a serum non-ceruloplasmin bound copper level of the subject using a copper speciation assay; and
controlling the subject's serum non-ceruloplasmin bound copper level to be in the range of from 25 to 130 ng/mL, preferably from 40 to 80 ng/mL.

The method typically comprises treating the Wilson's Disease by administration of a therapeutically effective amount of a copper (II) chelator. In some embodiments the copper (II) chelator is trientine tetrahydrochloride or D-penicillamine, preferably trientine tetrahydrochloride.

Therefore the present invention also provides a method of treating Wilson's Disease in a subject, wherein the method comprises
administering to the subject a therapeutically effective dose of a copper (II) chelator,
measuring a serum non-ceruloplasmin bound copper level of the subject using a copper speciation assay; and
controlling the subject's serum non-ceruloplasmin bound copper level to be in the range of from 25 to 130 ng/mL, preferably from 40 to 80 ng/mL.

### DESCRIPTION OF RELATED ART

### Brief Description of the Figures

FIG. 1: Representative LC-ICP-MS chromatogram of a healthy volunteer

FIG. 2: Representative LC-ICP-MS chromatogram of a Wilson's Disease patient

FIG. 3: Schematic of clinical trial study of Example 3

FIG. 4: Mean serum NCC±95% CI (intention to treat population) as determined in the clinical trial study of Example 3

FIG. 5: Mean 24-hr UCE±95% CI (intention to treat population) as determined in the clinical trial study of Example 3

FIG. 6: Shapiro-Wilk Test plot of NCC levels of healthy volunteers

FIG. 7: Bland Altman Plot of paired NCC-EDTA and NCC-CuSP values

### DETAILED DESCRIPTION OF THE INVENTION

### Treatments for Wilson's Disease

Any compounds which are useful in treating Wilson's Disease can be used in the methods described herein, for example methods of treating and assessing subjects who have, or who may have, Wilson's Disease. Typically, a compound which is useful in treating Wilson's Disease is a copper (II) chelator. For example, the compound may be trientine tetrahydrochloride, trientine dihydrochloride or D-penicillamine. Typically, the compound is trientine tetrahydrochloride or D-penicillamine. Preferably, the compound is trientine tetrahydrochloride.

As used herein, trientine tetrahydrochloride (TETA 4HCl) includes any amorphous or crystalline form of TETA 4HCl. In a preferred embodiment, the trientine tetrahydrochloride is a crystalline form of trientine tetrahydrochloride. Preferably, the trientine tetrahydrochloride is a crystalline form as described in WO 2019/211464. Thus, preferably the trientine tetrahydrochloride is, or comprises trientine tetrahydrochloride Form B. Preferably the trientine tetrahydrochloride comprises at least 90%, more preferably at least 95% of trientine tetrahydrochloride Form B.

Compounds referred to herein may be administered to a subject as a pharmaceutical composition comprising the compound together with one or more pharmaceutically acceptable carriers or diluents. The pharmaceutical composition may take any suitable form, but is preferably an oral dosage form. For example, the composition may take the form of a tablet, a capsule, a powder, a semisolid, a sustained release formulation, a solution, a suspension or any other appropriate composition. Tablets, capsules and powders, in particular tablets, are preferred. In alternative embodiments, the compositions are administered parenterally, for example subcutaneously or intravenously.

Suitable pharmaceutically acceptable carriers for the preparation of oral dosage forms include, for example, solubilising agents, e.g. cyclodextrins or modified cyclodextrins; diluents, e.g. lactose, dextrose, saccharose, cellulose, corn starch or potato starch; lubricants, e.g. silica, talc, stearic acid, magnesium or calcium stearate, and/or polyethylene glycols; binding agents; e.g. starches, tragacanth gums, gelatin, syrup, acacia, sorbitol, methylcellulose, carboxymethylcellulose or polyvinyl pyrrolidone; disaggregating agents, e.g. starch, alginic acid, alginates or sodium starch glycolate; effervescing mixtures; dyestuffs; sweeteners; wetting agents, such as lecithin, polysorbates, laurylsulphates; and, in general, non-toxic and pharmacologically inactive substances used in pharmaceutical formulations. Such pharmaceutical preparations may be manufactured in a known manner, for example, by means of mixing.

Typically, compounds which are useful in treating Wilson's Disease are used in the methods described herein by administering at least one therapeutically effective dose of the compound to a subject. It will be understood that the specific dose level for any particular subject will depend upon a variety of factors including the activity of the specific compound employed, the age, body weight, general health, sex, diet, time of administration, route of administration, rate of excretion, drug combination and the severity of the particular disease undergoing treatment. Optimum dose levels and frequency of dosing will usually be determined by clinical trial. As used herein, a therapeutically effective dose refers to a dose that is effective for producing some desired therapeutic effect, such as treating Wilson's Disease or treating or ameliorating one or more symptoms of Wilson's Disease.

A typical daily dose of trientine tetrahydrochloride or trientine dihydrochloride is up to 50 mg per kg of body weight, for example from 0.001 to 50 mg per kg of body weight, according to the age, weight and conditions of the subject to be treated, the type and severity of the disease and the frequency and route of administration. Typically, daily dosage levels of trientine tetrahydrochloride or trientine dihydrochloride are equivalent to from 300 to 1800 mg/day, preferably from 450 to 1200 mg/day, from 600 to 1200 mg/day, from 750 to 1200 mg/day, or from 750 to 1050 mg/day of trientine free base. Trientine free base is the active chelator in the case of administering trientine tetrahydrochloride or trientine dihydrochloride. Daily dosage levels are measured in terms of the amount of active chelator administered. Preferably, daily dosage levels of trientine tetrahydrochloride are from 300 to 1800 mg/day, from 450 to 1200 mg/day or more preferably from 450 to 975 mg/day of trientine free base. Preferably, daily dosage levels of trientine dihydrochloride are from 300 to 1800 mg/day, or from 450 to 1500 mg/day, or more preferably from 650 to 1350 mg/day of trientine free base/day. However, as described herein, the dosage level of trientine tetrahydrochloride or trientine dihydrochloride may be adjusted outside this range in order to control the serum NCC level of the subject to within a specific range. Typically, where dosages are adjusted, the maximum amount of active chelator administered is no more than 2000 mg/day, preferably no more than 1800 mg/day of trientine free base. For example a dose of trientine tetrahydrochloride or trientine dihydrochloride may be adjusted to a dose within the range of from 50 to 2000 mg/day of trientine free base.

Typical daily doses of D-penicillamine are well-known in the art. Typically, daily dosage levels of D-penicillamine are from 225 to 2000 mg/day, or from 500 to 1500 mg/day, or from 750 to 1500 mg/day, preferably from 750 to 1500 mg/day. However, as described herein, the dosage level of D-penicillamine may be adjusted be adjusted outside this range in order to control the serum NCC level of the subject to within a specific range. Typically, where dosages are adjusted, the maximum amount of D-penicillamine administered is no more than 2500 mg/day, preferably no more than 2000 mg/day. For example a dose of D-penicillamine may be adjusted to a dose within the range of from 50 to 2500 mg/day.

Where adjustment to dosages outside the usual daily dosage range is required, this may be a temporary adjustment, with the dosage being returned to the above-specified preferred dosing range once the patient's serum NCC level has been controlled.

Compounds that are useful in treating Wilson's Disease are typically administered to a subject at least once daily, for example once, twice or three times a day. Preferably, the compounds are administered at least twice daily. The compounds are typically administered at least 1 hour before food intake or at least 2 hours after food intake.

### Treating and Monitoring Wilson's Disease

The present invention relates to the treatment and/or monitoring of Wilson's Disease in a subject, where the treatment and/or monitoring involve measuring a serum non-ceruloplasmin (NCC) level of the subject and controlling the serum NCC level of the subject to be in a specified range. In this embodiment, the subject is typically a Wilson's Disease patient, i.e. a subject suffering from Wilson's Disease. A subject suffering from Wilson's Disease may require de-coppering treatment, i.e. where toxic levels of copper have built up in the body and need to be removed. Alternatively, a subject suffering from Wilson's Disease may require maintenance therapy, i.e. where the copper levels in the body are not at a toxic level but need to be controlled appropriately. Typically, a subject suffering from Wilson's Disease who is treated and/or monitored using the methods discussed below requires maintenance therapy.

Measuring serum NCC levels of a subject is typically carried out two or more times during the subject's treatment for Wilson's Disease. Where serum NCC levels are measured two or more times, this is described herein as monitoring the subject's serum NCC levels. Preferably, the subject's serum NCC levels are measured on a regular basis, for example a subject's serum NCC levels may be measured at least once every year, at least once every 9 months or at least once every 6 months, preferably at least once every 6 months during their treatment for Wilson's Disease. However, the subject's serum NCC levels may be measured more frequently if required, for example if the subject's treatment for Wilson's Disease has been adjusted (e.g. the identity of the copper (II) chelator administered to the subject has been changed, or the dose of the copper (II) chelator has been changed). In that case, the subject's serum NCC levels may be measured at least once every 3 months or at least once every month.

Controlling serum NCC levels of a subject to be in a specified range, or maintaining serum NCC levels of a subject in a specified range, encompasses methods in which some action needs to be taken to bring the serum NCC level of the subject into the specified range, as well as methods in which the serum NCC level of the subject is determined to already be within the specified range and so no action needs to be taken to bring the serum NCC level into the specified range. Controlling serum NCC levels of a subject to be in a specified range, or maintaining serum NCC levels of a subject in a specified range, also encompasses methods in which the serum NCC level of the subject falls inside the specified range for a period of time but which subsequently falls outside the specified range, in response to which action is taken to bring the serum NCC level back into the specified range. Typically, taking action to bring the serum NCC level of a subject into the specified range involves adjusting the dose of a copper (II) chelator that is administered to the subject. Preferably, the copper (II) chelator is trientine tetrahydrochloride or D-penicillamine, more preferably trientine tetrahydrochloride.

Typically, if the serum non-ceruloplasmin bound copper level of the subject is determined to be below the lower limit of the specified range, (for example, below 40 ng/mL) then the dose of copper (II) chelator that is administered to the subject will be reduced. Alternatively, if the serum non-ceruloplasmin bound copper level of the subject is determined to be above the upper limit of the specified range, (for example, above 80 ng/mL) then the dose of copper (II) chelator that is administered to the subject will be increased. Typically, the dose of copper (II) chelator is reduced or increased by 75 to 375 mg/day (of active chelator), preferably 150 to 300 mg/day (of active chelator). Preferably, the copper (II) chelator is trientine tetrahydrochloride or D-penicillamine, more preferably trientine tetrahydrochloride.

Following any dose adjustment (i.e. dose titration) that is required as part of the control of serum NCC levels, if following a further measurement the subject's serum NCC levels have returned to within the specified range, the dosage may be further adapted, for example by returning to the original dosage level. For example, if the dose has been increased following an increase in serum NCC levels, once the serum NCC levels have returned to the specified range, the dose may be reduced to the original dosage level. Similarly, if the dose has been reduced following a reduction in serum NCC levels, should the serum NCC levels return to the specified range, the dose may be increased to the original dosage level.

Accordingly, the present invention provides a copper (II) chelator for use in a method of treating Wilson's Disease in a subject, wherein the method comprises measuring a serum non-ceruloplasmin bound copper level of the subject; and controlling the subject's serum non-ceruloplasmin bound copper level to be in the range of from 25 to 130 ng/mL, preferably from 40 to 80 ng/mL. Typically, the copper (II) chelator is trientine tetrahydrochloride or D-penicillamine. In a preferred embodiment, the present invention provides trientine tetrahydrochloride for use in a method of treating Wilson's Disease in a subject, wherein the method comprises measuring a serum non-ceruloplasmin bound copper level of the subject; and controlling the subject's serum non-ceruloplasmin bound copper level to be in the range of from 25 to 130 ng/mL, preferably from 40 to 80 ng/mL.

The present invention also provides a copper (II) chelator for use in a method of monitoring a subject undergoing treatment for Wilson's Disease by administration of the copper (II) chelator, wherein the method comprises measuring a serum non-ceruloplasmin bound copper level of the subject; and controlling the subject's serum non-ceruloplasmin bound copper level to be in the range of from 25 to 130 ng/mL, preferably from 40 to 80 ng/mL. Typically, the copper (II) chelator is trientine tetrahydrochloride or D-penicillamine. In a preferred embodiment, the present invention provides trientine tetrahydrochloride for use in a method of monitoring a subject undergoing treatment for Wilson's Disease by administration of trientine tetrahydrochloride, wherein the method comprises measuring a serum non-ceruloplasmin bound copper level of the subject; and controlling the subject's serum non-ceruloplasmin bound copper level to be in the range of from 25 to 130 ng/mL, preferably from 40 to 80 ng/mL.

The present invention also relates to the treatment of Wilson's Disease in a subject, wherein the subject is treated with a copper (II) chelator and the serum NCC level of the subject is assessed during or after the treatment to determine if it is within a pre-determined range. In this embodiment, the subject is typically a Wilson's Disease patient, i.e. a subject suffering from Wilson's Disease.

The present invention therefore provides a copper (II) chelator for use in a method of treating Wilson's Disease in a subject, the method comprising:
a) administering to the subject a therapeutically effective dose of the copper (II) chelator;
b) measuring a serum non-ceruloplasmin bound copper level of the subject after administration of the dose of the copper (II) chelator using a copper speciation assay; and
c) determining whether the serum non-ceruloplasmin bound copper level of the subject after administration of the dose of the copper (II) chelator is within a range of from 25 to 130 ng/mL, preferably from 40 to 80 ng/mL.

Typically, the compound is trientine tetrahydrochloride or D-penicillamine. Preferably, the present invention provides trientine tetrahydrochloride for use in a method of treating Wilson's Disease in a subject, the method comprising:
a) administering to the subject a therapeutically effective dose of trientine tetrahydrochloride;
b) measuring a serum non-ceruloplasmin bound copper level of the subject after administration of the dose of trientine tetrahydrochloride using a copper speciation assay; and
c) determining whether the serum non-ceruloplasmin bound copper level of the subject after administration of the dose of trientine tetrahydrochloride is within a range of from 25 to 130 ng/mL, preferably from 40 to 80 ng/mL.

The present invention also provides the use of a copper (II) chelator in the manufacture of a medicament for use in the treatment of Wilson's Disease in a subject, the treatment comprising:
a) administering to the subject a therapeutically effective dose of the copper (II) chelator;
b) measuring a serum non-ceruloplasmin bound copper level of the subject after administration of the dose of the copper (II) chelator using a copper speciation assay; and
c) determining whether the serum non-ceruloplasmin bound copper level of the subject after administration of the dose of the copper (II) chelator is within a range of from 25 to 130 ng/mL, preferably from 40 to 80 ng/mL,
preferably wherein the copper (II) chelator is trientine tetrahydrochloride.

The present invention also provides a method of treating Wilson's Disease in a subject, the method comprising:
a) administering to the subject a therapeutically effective dose of a copper (II) chelator;
b) measuring a serum non-ceruloplasmin bound copper level of the subject after administration of the dose of the copper (II) chelator using a copper speciation assay; and
c) determining whether the serum non-ceruloplasmin bound copper level of the subject after administration of the dose of the copper (II) chelator is within a range of from 25 to 130 ng/mL, preferably from 40 to 80 ng/mL,
preferably wherein the copper (II) chelator is trientine tetrahydrochloride.

The methods described herein also allow for dose adjustment to optimise the treatment of the subject. For example, if the serum NCC level is found to be within the pre-determined range, then no changes to the treatment may be necessary. If the serum NCC level is found to be outside of the pre-determined range, then the treatment may be modified (for example, via dose adjustment) with the aim of bringing the serum NCC level into the pre-determined range.

Therefore, the present invention provides a copper (II) chelator for use in a method of treating Wilson's Disease in a subject, the method comprising:
a) administering to the subject at least a first and a further therapeutically effective dose of the copper (II) chelator;
b) measuring a serum non-ceruloplasmin bound copper level of the subject after administration of the first dose of the copper (II) chelator using a copper speciation assay;
c) determining whether the serum non-ceruloplasmin bound copper level of the subject after administration of the first dose of the copper (II) chelator is within a range of from 25 to 130 ng/mL, preferably from 40 to 80 ng/mL; and either
d) if the serum non-ceruloplasmin bound copper level of the subject after administration of the first dose of the copper (II) chelator is not within the range, modifying the further dose of the copper (II) chelator compared to the first dose; or
e) if the serum non-ceruloplasmin bound copper level of the subject after administration of the first dose of the copper (II) chelator is within the range, keeping the further dose the same as the first dose.

The copper (II) chelator may be trientine tetrahydrochloride or D-penicillamine. In a preferred embodiment, the present invention provides trientine tetrahydrochloride for use in a method of treating Wilson's Disease in a subject, the method comprising:
a) administering to the subject at least a first and a further therapeutically effective dose of trientine tetrahydrochloride;
b) measuring a serum non-ceruloplasmin bound copper level of the subject after administration of the first dose of trientine tetrahydrochloride using a copper speciation assay;
c) determining whether the serum non-ceruloplasmin bound copper level of the subject after administration of the first dose of trientine tetrahydrochloride is within a range of from 25 to 130 ng/mL, preferably from 40 to 80 ng/mL; and either
d) if the serum non-ceruloplasmin bound copper level of the subject after administration of the first dose of trientine tetrahydrochloride is not within the range, modifying the further dose of trientine tetrahydrochloride compared to the first dose; or
e) if the serum non-ceruloplasmin bound copper level of the subject after administration of the first dose of trientine tetrahydrochloride is within the range, keeping the further dose the same as the first dose.

The present invention also provides the use of a copper (II) chelator in the manufacture of a medicament for use in the treatment of Wilson's Disease in a subject, the treatment comprising:
a) administering to the subject at least a first and a further therapeutically effective dose of the copper (II) chelator;
b) measuring a serum non-ceruloplasmin bound copper level of the subject after administration of the first dose of the copper (II) chelator using a copper speciation assay;
c) determining whether the serum non-ceruloplasmin bound copper level of the subject after administration of the first dose of the copper (II) chelator is within a range of from 25 to 130 ng/mL, preferably from 40 to 80 ng/mL; and either
d) if the serum non-ceruloplasmin bound copper level of the subject after administration of the first dose of the copper (II) chelator is not within the range, modifying the further dose of the copper (II) chelator compared to the first dose; or
e) if the serum non-ceruloplasmin bound copper level of the subject after administration of the first dose of the copper (II) chelator is within the range, keeping the further dose the same as the first dose,
preferably wherein the copper (II) chelator is trientine tetrahydrochloride.

The present invention also provides a method of treating Wilson's Disease in a subject, the method comprising
a) administering to the subject at least a first and a further therapeutically effective dose of a copper (II) chelator;
b) measuring a serum non-ceruloplasmin bound copper level of the subject after administration of the first dose of the copper (II) chelator using a copper speciation assay;
c) determining whether the serum non-ceruloplasmin bound copper level of the subject after administration of the first dose of the copper (II) chelator is within a range of from 25 to 130 ng/mL, preferably from 40 to 80 ng/mL; and either
d) if the serum non-ceruloplasmin bound copper level of the subject after administration of the first dose of the copper (II) chelator is not within the range, modifying the further dose of the copper (II) chelator compared to the first dose; or
e) if the serum non-ceruloplasmin bound copper level of the subject after administration of the first dose of the copper (II) chelator is within the range, keeping the further dose the same as the first dose,
preferably wherein the copper (II) chelator is trientine tetrahydrochloride.

When the method comprises administering to the subject at least a first and a further therapeutically effective dose of a copper (II) chelator, if the serum non-ceruloplasmin bound copper level of the subject after administration of the first dose is not within a specified range, the further dose may be modified compared to the first dose.

Typically, if the serum non-ceruloplasmin bound copper level of the subject after administration of the first dose of the copper (II) chelator is below the lower limit of the specified range, (for example, below 40 ng/mL) then the further dose is reduced compared to the first dose. In a preferred embodiment where the copper (II) chelator is trientine tetrahydrochloride, if the serum non-ceruloplasmin bound copper level of the subject after administration of the first dose of trientine tetrahydrochloride is below the lower limit of the specified range (for example, below 40 ng/mL), the further dose is reduced compared to the first dose.

Typically, if the serum non-ceruloplasmin bound copper level of the subject after administration of the first dose is above the upper limit of the specified range, (for example, above 80 ng/mL) then the further dose is increased compared to the first dose. In a preferred embodiment where the copper (II) chelator is trientine tetrahydrochloride, if the serum non-ceruloplasmin bound copper level of the subject after administration of the first dose of trientine tetrahydrochloride is above the upper limit of the specified range (for example, above 80 ng/mL), the further dose is increased compared to the first dose.

The amount the further dose is adjusted by will typically depend on the level of the first dose, and/or how far a subject's serum NCC levels are outside the specified range. Typically, the further dose of the compound is adjusted (increased or reduced) by 75 to 375 mg/day of active chelator, preferably by 150 to 300 mg/day. Typically, the further dose of the compound is increased by 150 to 300 mg/day based on the amount of active chelator compared to the first dose or the further dose of the compound is reduced by 150 to 300 mg/day based on the amount of active chelator compared to the first dose. The active chelator is the compound which actually chelates copper (II) in the body. For trientine tetrahydrochloride and trientine dihydrochloride, the active chelator is trientine free base. For D-penicillamine, the active chelator is D-penicillamine. For example, the further dose of trientine tetrahydrochloride may be increased or reduced by 75 to 375 mg/day, preferably 150 to 300 mg/day, based on the amount of trientine free base/day. Alternatively, the further dose of D-penicillamine may be increased or reduced by 75 to 375 mg/day, preferably 150 to 300 mg/day. Alternatively, the further dose of trientine dihydrochloride may be increased or reduced by 75 to 375 mg/day, preferably 150 to 300 mg/day, based on the amount of trientine free base/day. In one embodiment, if the first dose is less than or equal to 1000 mg/day of active chelator then the further dose of the compound is increased or reduced by about 150 mg of trientine free base/day compared to the first dose, and if the first dose is greater than 1000 mg/day of active chelator then the further dose of the compound is increased or reduced by about 300 mg of trientine free base/day compared to the first dose.

In some embodiments, the subject is being treated with TETA 4HCl at a dose of from 450 to 600 mg of trientine free base/day, and (i) if the subject's serum NCC levels are determined to be below 40 ng/mL, the dose of TETA 4HCl is reduced by 150 mg of trientine free base/day to 300 to 450 mg of trientine free base/day, or (ii) if the subject's serum NCC levels are determined to be within the range from 40 to 80 ng/mL, treatment is continued at the same dose of 450 to 600 mg of trientine free base/day, or (iii) if the subject's serum NCC levels are determined to be above 80 ng/mL, the dose of TETA 4HCl is increased by 150 mg of trientine free base/day to 600 to 750 mg of trientine free base/day. In some embodiments, the subject is being treated with TETA 4HCl at a dose of from 450 to 600 mg of trientine free base/day, and (i) if the subject's serum NCC levels are determined to be below 40 ng/mL, the dose of TETA 4HCl is reduced by 75 mg of trientine free base/day to 375 to 525 mg of trientine free base/day, or (ii) if the subject's serum NCC levels are determined to be within the range from 40 to 80 ng/mL, treatment is continued at the same dose of 450 to 600 mg of trientine free base/day, or (iii) if the subject's serum NCC levels are determined to be above 80 ng/mL, the dose of TETA 4HCl is increased by 75 mg of trientine free base/day to 525 to 675 mg of trientine free base/day.

In some embodiments, the subject is being treated with TETA 4HCl at a dose of from 600 to 750 mg of trientine free base/day, and (i) if the subject's serum NCC levels are determined to be below 40 ng/mL, the dose of TETA 4HCl is reduced by 150 mg of trientine free base/day to 450 to 600 mg of trientine free base/day, or (ii) if the subject's serum NCC levels are determined to be within the range from 40 to 80 ng/mL, treatment is continued at the same dose of 600 to 750 mg of trientine free base/day, or (iii) if the subject's serum NCC levels are determined to be above 80 ng/mL, the dose of TETA 4HCl is increased by 150 mg of trientine free base/day to 750 to 900 mg of trientine free base/day.

In some embodiments, the subject is being treated with TETA 4HCl at a dose of from 750 to 1000 mg of trientine free base/day, and (i) if the subject's serum NCC levels are determined to be below 40 ng/mL, the dose of TETA 4HCl is reduced by 150 mg of trientine free base/day to 600 to 850 mg of trientine free base/day, or (ii) if the subject's serum NCC levels are determined to be within the range from 40 to 80 ng/mL, treatment is continued at the same dose of 750 to 1000 mg of trientine free base/day, or (iii) if the subject's serum NCC levels are determined to be above 80 ng/mL, the dose of TETA 4HCl is increased by 150 mg of trientine free base/day to 900 to 1150 mg of trientine free base/day. In some embodiments, the subject is being treated with TETA 4HCl at a dose of from 750 to 1000 mg of trientine free base/day, and (i) if the subject's serum NCC levels are determined to be below 40 ng/mL, the dose of TETA 4HCl is reduced by 225 mg of trientine free base/day to 525 to 775 mg of trientine free base/day, or (ii) if the subject's serum NCC levels are determined to be within the range from 40 to 80 ng/mL, treatment is continued at the same dose of 750 to 1000 mg of trientine free base/day, or (iii) if the subject's serum NCC levels are determined to be above 80 ng/mL, the dose of TETA 4HCl is increased by 225 mg of trientine free base/day to 975 to 1225 mg of trientine free base/day.

In some embodiments, the subject is being treated with TETA 4HCl at a dose of from 1000 to 1200 mg of trientine free base/day, and (i) if the subject's serum NCC levels are determined to be below 40 ng/mL, the dose of TETA 4HCl is reduced by 300 mg of trientine free base/day to 700 to 900 mg of trientine free base/day, or (ii) if the subject's serum NCC levels are determined to be within the range from 40 to 80 ng/mL, treatment is continued at the same dose of 1000 to 1200 mg of trientine free base/day, or (iii) if the subject's serum NCC levels are determined to be above 80 ng/mL, the dose of TETA 4HCl is increased by 300 mg of trientine free base/day to 1300 to 1500 mg of trientine free base/day.

When the method comprises administering to the subject at least a first and a further therapeutically effective dose of a copper (II) chelator, if the serum non-ceruloplasmin bound copper level of the subject after administration of the first dose of the copper (II) chelator is within a specified range, the further dose is kept the same as the first dose. The specified range may be any range as described herein. In a preferred embodiment where the copper (II) chelator is trientine tetrahydrochloride, if the serum non-ceruloplasmin bound copper level of the subject after administration of the first dose of trientine tetrahydrochloride is within the specified range (for example, from 40 to 80 ng/mL), then the further dose of trientine tetrahydrochloride is the same as the first dose.

In each of the methods and uses described herein, the specified or pre-determined range of serum NCC levels is typically from 25 to 130 ng/mL. Preferably, the range is from 40 to 80 ng/mL, or from 45 to 80 ng/mL, or from 46 to 80 ng/mL, more preferably from 40 to 80 ng/mL. The range may be from 40 to 310 ng/mL, from 40 to 150 ng/mL, from 50 to 150 ng/mL, or from 50 to 110 ng/mL, preferably from 50 to 110 ng/mL. In one embodiment, the range is from 40 to 150 ng/mL. In one embodiment, the range is from 50 to 110 ng/mL. Alternatively, the range is from 25 to 150 ng/mL, or from 25 to 100 ng/mL. The range may be have an upper limit of less than 50 ng/mL, for example the range may be from 25 to 49 ng/mL, or from 40 to 49 ng/mL.

It has been found that maintaining serum NCC levels within these ranges provides very effective control of a subject's Wilson's Disease. In particular, it is thought that maintaining the NCC level of a Wilson's Disease patient within the range of from 25 to 130 ng/mL, and particularly from 40 to 80 ng/mL (for example 45 to 80 ng/mL or 25 to 49 ng/mL), as determined using the copper speciation assay described herein, is important because a Wilson's Disease patient typically has pre-existing copper mediated tissue injury. Therefore, there may be clinical benefits associated with keeping the NCC level of such a patient tightly controlled within the specific ranges described above. In comparison, healthy patients can typically tolerate a wider range of NCC levels (for example, a healthy patient can tolerate NCC levels in the region of 200-300 ng/mL) without any negative clinical effect.

In the methods described herein, the serum NCC level of the subject may be measured after administration of a dose of a copper (II) chelator. It will be appreciated that a subject typically receives multiple doses of a copper (II) chelator during treatment, and that measurement of the serum NCC level of a subject may occur after one, two, or multiple doses of the copper (II) chelator. Subjects are typically dosed at least daily during treatment, preferably twice daily, and the serum NCC level of the subject may be measured a certain time after the administration of a nominal “first” dose. It will be appreciated that this “first” dose may not actually be the first dose of copper (II) chelator that the subject receives, but is used as a reference dose for defining when the measurement of serum NCC level is taken. A “further” dose may also be administered to the subject after the assessment of the serum NCC levels. It will be appreciated that the subject will typically receive multiple additional doses between the “first” and “further” doses, but that the term “further” dose is used to identify a dose that is administered with any necessary dose adjustment, if such dose adjustment is deemed to be required following the serum NCC measurement.

Therefore typically, the serum NCC level of the subject is measured at least one day, at least one week, at least one month, at least three months, at least six months or at least one year after administration of the first dose. Preferably, the serum NCC level of the subject is measured at least one month, at least three months, or at least six months after administration of the first dose.

### Assessing Wilson's Disease

Determining whether the serum NCC level of a subject is within a specified range, and in particular above a specific value, may also be used to assess subjects who are susceptible to Wilson's Disease and who may or may not be displaying symptoms of Wilson's Disease. For example the subjects may be subjects who are pre-disposed (e.g. genetically pre-disposed) to Wilson's Disease and who may or may not be displaying symptoms of Wilson's Disease. The method is particularly useful in assessing those who are susceptible to Wilson's Disease, for example who are pre-disposed to Wilson's Disease (e.g. genetically pre-disposed) to Wilson's Disease, but who are not displaying symptoms of Wilson's Disease.

Therefore, the present invention also provides a method of assessing a subject who is susceptible to Wilson's Disease, wherein the method comprises:
a) measuring a serum non-ceruloplasmin bound copper level of the subject using a copper speciation assay; and
b) determining whether the serum non-ceruloplasmin bound copper level of the subject is above a specified range or value as set out herein, in particular above 150 ng/mL).

Subjects who are susceptible to Wilson's Disease are typically those who are genetically pre-disposed to Wilson's Disease. The gene responsible for Wilson's Disease, ATP7B, is highly expressed in the liver, kidney, and placenta, and has been identified on chromosome 13 (Tanzi, R., Petrukhin, K. et al. (1993). “The Wilson disease gene is a copper transporting ATPase with homology to the Menkes disease gene.” Nat Genet 5(4): 344-350). More than 500 distinct mutations have been described in this gene, from which 380 have a confirmed role in the pathogenesis of the disease. However, it is well reported that individual gene mutations do not reliably lead to a clinical diagnosis of Wilson's Disease, which will also be affected by environmental factors and epigenetics.

The specified range may be any range disclosed herein. However, in general the serum NCC level of a subject who is susceptible to Wilson's Disease, and who may benefit from treatment for Wilson's Disease, is above the specified ranges discussed herein, rather than below. Therefore, assessing a subject who is susceptible to Wilson's Disease may typically involve determining whether the subject's serum NCC level is above a specified value, rather than within a specified range. The specified value may be 80 ng/mL, or 100 ng/mL, or 110 ng/mL, or 120 ng/mL, or 130 ng/mL, or 140 ng/mL, or 150 ng/mL. Preferably the specified value is 150 ng/mL.

The methods of assessing a subject who is susceptible to Wilson's Disease may be used in a method of diagnosing a subject as having Wilson's Disease. Thus, disclosed herein is a method of diagnosing a subject as having Wilson's Disease, wherein the method comprises:
a) measuring a serum non-ceruloplasmin bound copper level of the subject using a copper speciation assay;
b) determining whether the serum non-ceruloplasmin bound copper level of the subject is above a specified range or value as set out herein, in particular above 150 ng/mL; and
c) diagnosing the subject as having Wilson's Disease if the serum non-ceruloplasmin bound copper level is above a specified range or value as set out herein, in particular above 150 ng/mL.

In one embodiment, the subject may be diagnosed as having Wilson's Disease if the serum non-ceruloplasmin bound copper level is above 120 ng/mL. In one embodiment, the subject may be diagnosed as having Wilson's Disease if the serum non-ceruloplasmin bound copper level is above 140 ng/mL.

Typically, the methods of assessment and diagnosis described herein comprise measuring a serum non-ceruloplasmin bound copper level of a serum sample obtained from a subject. This step is performed in vitro, i.e. the methods of assessment and diagnosis comprise measuring in vitro a serum non-ceruloplasmin bound copper level of a serum sample obtained from a subject. The methods may comprise measuring a serum non-ceruloplasmin bound copper level of a serum sample previously obtained from a subject. In one embodiment, the methods comprise measuring in vitro a serum non-ceruloplasmin bound copper level of a serum sample previously obtained from a subject.

A subject who has been assessed and/or diagnosed according to the methods described herein may then be treated using the methods of treatment described herein. For example, the subject may then be treated by administering a copper (II) chelator such as trientine tetrahydrochloride, trientine dihydrochloride or D-penicillamine. Preferably, the subject may be treated by administration of trientine tetrahydrochloride or D-penicillamine, more preferably by administration of trientine tetrahydrochloride.

Therefore, in one embodiment the present invention provides a method of assessing and treating a subject who is susceptible to Wilson's Disease, wherein the method comprises:
a) measuring a serum non-ceruloplasmin bound copper level of the subject using a copper speciation assay;
b) determining whether the serum non-ceruloplasmin bound copper level of the subject is above a specified range or value as set out herein, in particular above 150 ng/mL; and
c) if the serum non-ceruloplasmin bound copper level of the subject is above a specified range or value as set out herein, in particular above 150 ng/mL, treating the subject for Wilson's Disease preferably by administration of a copper (II) chelator, preferably wherein the copper (II) chelator is trientine tetrahydrochloride.

In one embodiment, the present invention provides a copper (II) chelator for use in a method of assessing and treating a subject who is susceptible to Wilson's Disease, wherein the method comprises:
a) measuring a serum non-ceruloplasmin bound copper level of the subject using a copper speciation assay;
b) determining whether the serum non-ceruloplasmin bound copper level of the subject is above a specified range or value as set out herein, in particular above 150 ng/mL; and
c) if the serum non-ceruloplasmin bound copper level of the subject is above a specified range or value as set out herein, in particular above 150 ng/mL, administering a therapeutically effective dose of the copper (II) chelator to the subject.

In a preferred embodiment, the present disclosure provides trientine tetrahydrochloride for use in a method of assessing and treating a subject who is susceptible to Wilson's Disease, wherein the method comprises:
a) measuring a serum non-ceruloplasmin bound copper level of the subject using a copper speciation assay;
b) determining whether the serum non-ceruloplasmin bound copper level of the subject is above a specified range or value as set out herein, in particular above 150 ng/mL; and
c) if the serum non-ceruloplasmin bound copper level of the subject is above a specified range or value as set out herein, in particular above 150 ng/mL, administering a therapeutically effective dose of trientine tetrahydrochloride to the subject.

The present invention also provides the use of a copper (II) chelator in the manufacture of a medicament for use in assessing and treating a subject who is susceptible to Wilson's Disease, wherein the assessment and treatment comprises:
a) measuring a serum non-ceruloplasmin bound copper level of the subject using a copper speciation assay;
b) determining whether the serum non-ceruloplasmin bound copper level of the subject is above a specified range or value as set out herein, in particular above 150 ng/mL; and
c) if the serum non-ceruloplasmin bound copper level of the subject is above a specified range or value as set out herein, in particular above 150 ng/mL, administering a therapeutically effective dose of the copper (II) chelator to the subject.

A subject who has been assessed and determined to require treatment with a copper (II) chelator according to these embodiments may then be treated with a copper (II) chelator as described herein. As a part of this treatment, the subject may have their serum NCC levels measured and controlled as described herein. Preferably the subject's serum NCC levels are measured two or more times during the treatment, i.e. they are monitored as described herein.

Thus, the methods of assessing and treating a subject who is susceptible to Wilson's Disease that are described herein may also be combined with the monitoring and dose adjustment steps that are described herein for monitoring and/or treating subjects with Wilson's Disease.

For example, in one embodiment the present invention provides a copper (II) chelator for use in a method of assessing and treating Wilson's Disease in a subject, the method comprising:
a) measuring a serum non-ceruloplasmin bound copper level of the subject using a copper speciation assay;
b) determining whether the serum non-ceruloplasmin bound copper level of the subject is above a specified range or value as set out herein, in particular above 150 ng/mL;
c) if the serum non-ceruloplasmin bound copper level of the subject is above a specified range or value as set out herein, in particular above 150 ng/mL, administering a therapeutically effective dose of the copper (II) chelator to the subject;
d) measuring a serum non-ceruloplasmin bound copper level of the subject after administration of the dose of the copper (II) chelator using a copper speciation assay; and
e) determining whether the serum non-ceruloplasmin bound copper level of the subject after administration of the dose of the copper (II) chelator is within a range of from 25 to 130 ng/mL, preferably from 40 to 80 ng/mL,
preferably wherein the copper (II) chelator is trientine tetrahydrochloride.

The present invention also provides a copper (II) chelator for use in a method of assessing and treating Wilson's Disease in a subject, the method comprising:
a) measuring a serum non-ceruloplasmin bound copper level of the subject using a copper speciation assay;
b) determining whether the serum non-ceruloplasmin bound copper level of the subject is above a specified range or value as set out herein, in particular above 150 ng/mL;
c) if the serum non-ceruloplasmin bound copper level of the subject is above a specified range or value as set out herein, in particular above 150 ng/mL, administering at least a first and a further therapeutically effective dose of the copper (II) chelator to the subject;
d) measuring a serum non-ceruloplasmin bound copper level of the subject after administration of the first dose of the copper (II) chelator using a copper speciation assay;
e) determining whether the serum non-ceruloplasmin bound copper level of the subject after administration of the dose of the copper (II) chelator is within a range of from 25 to 130 ng/mL, preferably from 40 to 80 ng/mL; and either
f) if the serum non-ceruloplasmin bound copper level of the subject after administration of the first dose of the copper (II) chelator is not within a range of from 25 to 130 ng/mL, preferably from 40 to 80 ng/mL, modifying the further dose of the copper (II) chelator compared to the first dose; or
g) if the serum non-ceruloplasmin bound copper level of the subject after administration of the first dose of the copper (II) chelator is within a range of from 25 to 130 ng/mL, preferably from 40 to 80 ng/mL, keeping the further dose the same as the first dose.

The present invention also provides the use of a copper (II) chelator in the manufacture of a medicament for use in the assessment and treatment of Wilson's Disease in a subject, the method comprising:
a) measuring a serum non-ceruloplasmin bound copper level of the subject using a copper speciation assay;
b) determining whether the serum non-ceruloplasmin bound copper level of the subject is above a specified range or value as set out herein, in particular above 150 ng/mL;
c) if the serum non-ceruloplasmin bound copper level of the subject is above a specified range or value as set out herein, in particular above 150 ng/mL, administering a therapeutically effective dose of the copper (II) chelator to the subject;
d) measuring a serum non-ceruloplasmin bound copper level of the subject after administration of the dose of the copper (II) chelator using a copper speciation assay; and
e) determining whether the serum non-ceruloplasmin bound copper level of the subject after administration of the dose of the copper (II) chelator is within a range of from 25 to 130 ng/mL, preferably from 40 to 80 ng/mL,
preferably wherein the copper (II) chelator is trientine tetrahydrochloride.

The present invention also provides the use of a copper (II) chelator in the manufacture of a medicament for use in the assessment and treatment of Wilson's Disease in a subject, the method comprising:
a) measuring a serum non-ceruloplasmin bound copper level of the subject using a copper speciation assay;
b) determining whether the serum non-ceruloplasmin bound copper level of the subject is above a specified range or value as set out herein, in particular above 150 ng/mL;
c) if the serum non-ceruloplasmin bound copper level of the subject is above a specified range or value as set out herein, in particular above 150 ng/mL, administering at least a first and a further therapeutically effective dose of the copper (II) chelator to the subject;
d) measuring a serum non-ceruloplasmin bound copper level of the subject after administration of the first dose of the copper (II) chelator using a copper speciation assay;
e) determining whether the serum non-ceruloplasmin bound copper level of the subject after administration of the dose of the copper (II) chelator is within a range of from 25 to 130 ng/mL, preferably from 40 to 80 ng/mL; and either
f) if the serum non-ceruloplasmin bound copper level of the subject after administration of the first dose of the copper (II) chelator is not within a range of from 25 to 130 ng/mL, preferably from 40 to 80 ng/mL, modifying the further dose of the copper (II) chelator compared to the first dose; or
g) if the serum non-ceruloplasmin bound copper level of the subject after administration of the first dose of the copper (II) chelator is within a range of from 25 to 130 ng/mL, preferably from 40 to 80 ng/mL, keeping the further dose the same as the first dose.

The present invention also provides a method of assessing and treating Wilson's Disease in a subject, the method comprising:
a) measuring a serum non-ceruloplasmin bound copper level of the subject using a copper speciation assay;
b) determining whether the serum non-ceruloplasmin bound copper level of the subject is above a specified range or value as set out herein, in particular above 150 ng/mL;
c) if the serum non-ceruloplasmin bound copper level of the subject is above a specified range or value as set out herein, in particular above 150 ng/mL, administering a therapeutically effective dose of a copper (II) chelator to the subject;
d) measuring a serum non-ceruloplasmin bound copper level of the subject after administration of the dose of the copper (II) chelator using a copper speciation assay; and
e) determining whether the serum non-ceruloplasmin bound copper level of the subject after administration of the dose of the copper (II) chelator is within a range of from 25 to 130 ng/mL, preferably from 40 to 80 ng/mL,
preferably wherein the copper (II) chelator is trientine tetrahydrochloride.

The present invention also provides a method of assessing and treating Wilson's Disease in a subject, the method comprising:
a) measuring a serum non-ceruloplasmin bound copper level of the subject using a copper speciation assay;
b) determining whether the serum non-ceruloplasmin bound copper level of the subject is above a specified range or value as set out herein, in particular above 150 ng/mL;
c) if the serum non-ceruloplasmin bound copper level of the subject is above a specified range or value as set out herein, in particular above 150 ng/mL, administering at least a first and a further therapeutically effective dose of a copper (II) chelator to the subject;
d) measuring a serum non-ceruloplasmin bound copper level of the subject after administration of the first dose of the copper (II) chelator using a copper speciation assay;
e) determining whether the serum non-ceruloplasmin bound copper level of the subject after administration of the dose of the copper (II) chelator is within a range of from 25 to 130 ng/mL, preferably from 40 to 80 ng/mL; and either
f) if the serum non-ceruloplasmin bound copper level of the subject after administration of the first dose of the copper (II) chelator is not within a range of from 25 to 130 ng/mL, preferably from 40 to 80 ng/mL, modifying the further dose of the copper (II) chelator compared to the first dose; or
g) if the serum non-ceruloplasmin bound copper level of the subject after administration of the first dose of the copper (II) chelator is within a range of from 25 to 130 ng/mL, preferably from 40 to 80 ng/mL, keeping the further dose the same as the first dose.

As used herein, the term “subject” may refer to human or animal subjects, in particular humans or mammals, typically humans.

As used herein, the term “treating” is to be understood as embracing treatment and/or amelioration and/or prevention of or reduction in aggravation/worsening of symptoms of a disease or condition, and may include reversing or reducing the symptoms, clinical signs, and underlying pathology of a disease or condition in a manner to improve or stabilise a subject's condition.

### Method of Measuring Serum Non-Ceruloplasmin Bound Copper

In the methods disclosed herein, the serum non-ceruloplasmin bound copper level is measured using a copper speciation assay. A copper speciation assay is an assay which can separate copper bound to different species in a sample, for example a serum sample. Typically, the copper speciation assay can separate the Cu-Ceruloplasmin and Cu-Albumin fractions in the sample. Preferably, the copper speciation assay can separate Cu-Ceruloplasmin, Cu-Albumin and Cu-α-2-macroglobulin fractions in the sample. A copper speciation assay does not include, for example, the EDTA method of determining serum NCC levels in which EDTA is used to bind to copper not bound to ceruloplasmin and uses filtration to distinguish high and low molecular weight copper environments. In one embodiment, the serum non-ceruloplasmin bound copper level is typically measured using the following steps:
i) determining the total serum copper level, preferably using ICP-MS (inductively coupled plasma mass spectrometry); and
ii) determining the serum ceruloplasmin-derived copper level using a copper speciation assay, preferably using a chromatographic method; and
iii) calculating the serum non-ceruloplasmin bound copper level by subtracting the value obtained in step ii) from the value obtained in step i).

The total serum copper level may be determined using any appropriate method, for example inductively coupled plasma atomic emission spectroscopy (ICP-AES) (also known as ICP Optical Emission Spectroscopy, ICP-OES), derivatisation techniques using fluorescence or colour tags, or ICP-MS. Preferably, the total serum copper level is determined using ICP-MS.

As used herein, a chromatographic method may be any chromatographic method that can separate copper bound to different species in a serum sample. Preferably, the chromatographic method is capable of separating Cu-Ceruloplasmin, Cu-Albumin and Cu-α-2-macroglobulin. In one embodiment, the chromatographic method is anion exchange chromatography, preferably anion exchange liquid chromatography and more preferably anion exchange LC-ICP-MS (liquid chromatography inductively coupled plasma mass spectrometry).

The copper speciation assay with anion exchange LC-ICP-MS typically separates and identifies at least three copper-containing serum fractions (Cu-Ceruloplasmin+Cu-Albumin+Cu-Void). The Cu-Void peak is thought to be copper associated with α-2-macroglobulin.

The serum NCC level may be determined by subtracting the amount of Cu-Ceruloplasmin from the total Cu serum level. Thus in one embodiment, the serum non-ceruloplasmin bound copper level is measured using the following steps:
i) determining the total serum copper level, preferably using ICP-MS;
ii) determining the serum ceruloplasmin-derived copper level using a copper speciation assay with anion exchange LC-ICP-MS; and
iii) calculating the serum non-ceruloplasmin bound copper level by subtracting the value obtained in step ii) from the value obtained in step i).

In a preferred embodiment, the serum ceruloplasmin-derived copper level is derived from the ICP-MS copper speciation assay using the following equations (1) and (2):

$$\% Peak area of Cu - Cp \text{:} ( \frac{Peak area of Cu - Cp}{( Peak area of all Cu - containing peaks} ) \times 100 \qquad ( 1 )$$

The relative concentration (ng/mL) of Cu-Cp may then calculated as follows, using the total serum copper level:

$$( \frac{Total serum Cu concentration ( \frac{ng}{mL} )}{100} ) \times \% Peak Area \qquad ( 2 )$$

Thus, in a preferred embodiment, the serum non-ceruloplasmin bound copper level is measured using the following steps:
i) determining the total serum copper level, preferably using ICP-MS;
ii) determining the serum ceruloplasmin-derived copper level using a copper speciation assay with anion exchange LC-ICP-MS, wherein the ceruloplasmin-derived copper level is calculated according to equations (1) and (2); and
iii) calculating the non-ceruloplasmin bound copper level by subtracting the value obtained in step ii) from the value obtained in step i).

In some embodiments of the invention, measurement of the serum NCC level of a subject (preferably using the copper speciation assay described herein) enables a subject to be assessed using fewer biochemical and clinical tests. Preferably, measurement of the serum NCC level of a subject using the copper speciation assay described herein is the only biochemical assay, most preferably it is the only clinical tool, used to assess or monitor the subject. For example, in the methods described herein the subject may not undergo a test for 24-hour urinary copper excretion. Alternatively or in addition, the subject may not undergo a ALT test. In some embodiments, the subject undergoes a single biochemical assay to assess or monitor their Wilson's Disease, wherein the single biochemical assay is the serum NCC assay described herein (wherein the biochemical assay preferably uses the copper speciation assay described herein). In some embodiments, clinical assessments of the subject may be carried out less frequently. In some embodiments, the subject is not assessed for the presence or absence of Kayser-Fleischer rings, and/or liver function, and/or liver copper levels, and/or neurological symptoms using the Unified Wilson's Disease Rating Scale. In embodiments, measurement of the serum NCC level of a subject (preferably using the copper speciation assay described herein) is the only clinical tool used to assess or monitor the subject.

The ability to accurately assess and/or monitor subjects using a single biochemical test, and fewer, or only one, clinical tool(s) means that patient assessment uses fewer resources, is quicker, and is less burdensome on the patient which typically results in greater patient compliance.

### EXAMPLES

### Example 1—Total Copper (Cu) Determination in Serum Samples by ICP-MS

Example 1 describes an ICP-MS assay for the quantification of copper in human serum.

### Sample Preparation and Reagents

All chemicals used in this study were reagent grade or better. Copper (Sigma Aldrich, batch BCBW9300, expiry 4 May 2021) and rhodium (Sigma Aldrich, batch: BCBW5968, expiry 22 Feb. 2021). Human serum was obtained from commercial sources.

Intermediate calibration and validation solutions were prepared by mixing copper commercial solution with water to give a nominal concentration of 200 μg/mL. The intermediate solutions were used fresh on the day of preparation then disposed. Working calibration and validation solutions were prepared by mixing intermediate calibration or validation stock solution with water to give nominal concentrations of 2.00, 5.00, 10.0, 20.0, 50.0, 100, 150 and 200 μg/mL. Working solutions were used fresh on the day of preparation then disposed.

Calibration samples were prepared by mixing surrogate matrix (1% EDTA/1% TMAH) with copper working solutions to give nominal concentrations of 20.0, 50.0, 100, 200, 500, 1000, 1500 and 2000 ng/mL A dual calibration line was run bracketing the validation samples. Serum blanks prepared without (matrix blank), and with internal standard (QC0), were included with each analytical run. Calibration samples were prepared between 17 Jun. 2020 to 17 Jul. 2020 and stored frozen at approximately −80° C. for no longer than 30 days and used within their documented stability period.

Validation standards were prepared by mixing human serum with copper working solutions to give nominal concentrations of approximately 20.0, 50.0, 1000, 1500 and 2000 ng/mL. Validation samples were prepared between 17 Jun. 2020 and 17 Jul. 2020 and stored frozen at approximately −80° C. for no longer than 25 days and used within their documented stability period.

### Assay Procedure

1.95 mL of rhodium internal standard solution containing 1% TMAH/1% EDTA was combined with 50 μL of human serum in a 15 mL polypropylene tube, capped and rotary mixed for at least 30 minutes. The processed sample was then analysed by ICP-MS on an Agilent 7900x inductively coupled plasma mass spectrometer.

### Instrumentation

An Agilent 7900x series ICP-MS was used to quantify copper (mass 63) relative to the rhodium (mass 105) internal standard, following infusion of the sample into the ICP-MS via an integrated sample introduction system (ISIS) and micromist nebulizer.

Data were acquired using MassHunter (version C.01.05 Agilent). Elemental count results were exported to Watson LIMS (Version 7.5 SP1, Thermo Scientific) in text file format. Watson LIMS was used to calculate standard curve parameters and concentration data for copper. The concentration and statistical data were generated by computerized techniques.

### Assay Specificity and Calibration

Copper has 2 stable isotopes (63Cu and 65Cu), of which 63Cu is the most abundant and therefore this isotope was used to quantify copper by ICP-MS. Rhodium possesses only one stable isotope 105Rh and this was used to quantify rhodium by ICP-MS.

Calibration curves for copper, over the range of 20.0 to 2000 ng/mL, were fitted to a linear model using a weighting of 1/concentration2.

### Results

The total copper level for each lot of human serum, along with the within-run precision is shown in Table 1 below.

TABLE 1
Mean copper concentrations and within-run
precision for each lot of human serum.

| | Mean | | | Between- | |
|---|---|---|---|---|---|
| Nominal | Calculated | Mean | Within-run | run | Total |
| Conc. | Conc | Difference | Precision | Precision | Precision |
| (ng/mL) | (ng/mL) | (%) | (CV %) | (CV %) | (CV %) |
| 16.5 | 16.8 | 1.82 | 9.63 | 0.00 | 9.47 |
| 46.3 | 46.9 | 1.30 | 6.02 | 4.07 | 7.27 |
| 1020 | 1020 | 0.00 | 1.91 | 3.02 | 3.58 |
| 1550 | 1530 | −1.29 | 1.67 | 1.06 | 1.97 |

Accuracy, within-run precision, between-run precision and total precision (assay variability), assessed on three separate occasions at 20.0, 50.0, 1000 and 1500 ng/mL, were within acceptance limits. The accuracy ranged from −1.29 to 1.83% of nominal values and the total precision ranged from 1.89 to 9.52%. The influence of endogenous components, demonstrated using six different lots of human serum (matrix variability), had no effect on the quantitation of copper. The maximum run size for this assay (assay robustness) was 200 injections per batch. The overall process efficiency of this method is 94.7 to 96.0%. Dilution studies demonstrated that concentrations up to 20000 ng/mL of copper could be reliably analysed when diluted into the calibration range.

### Example 2—Cu Speciation Analysis by Anion Exchange HPLC-ICP-MS of the Whole Serum

Example 2 describes the LC-ICP-MS assay for the speciation of copper in human serum samples, which when combined with total serum copper concentration data can generate a ceruloplasmin-bound (and non-ceruloplasmin bound) copper relative concentration.

### Sample Preparation and Reagents

All chemicals used in this study were reagent grade or better. Human serum was obtained from commercial sources. Each lot of serum was analysed (n=6) for total serum copper content by ICP-MS in accordance with the method of Example 1. Ceruloplasmin (Sigma Aldrich, batch: 110M7012, expiry 15 Jun. 2022) and albumin (Sigma Aldrich, batch: SLBV3642, expiry 18 Jun. 2022) were obtained from commercial sources for use as reference standards.

### Assay Procedure

The samples were defrosted and equilibrated to room temperature prior to use, and vortex mixed for approximately 5 seconds. 80 μL of 50 mM Tris-HCl (pH 7.4) buffer solution was combined with 20 μL of the clinical sample, blank or reference standard in a 0.5 mL Eppendorf tube (protein low bind). The diluted sample (100 μL) was transferred to a 0.3 mL polypropylene LC vial and sealed with a screw cap. An aliquot (50 μL) of each processed sample was then analysed by LC-ICP-MS on an Agilent 8900x inductively coupled plasma mass spectrometer.

### Instrumentation

Speciation analysis of Cu-containing species in the serum samples was performed by an anion exchange high performance liquid chromatography (AE HPLC) column (GE Healthcare MonoQ 5/50 GL, stationary phase (10 μm; 5×50 mm)) with an ammonium acetate gradient (0-250 mM) for 20 min. A flow rate of 1.0 mL/min was used. The chromatographic separation was performed at 25° C. Detection of the eluting Cu species was achieved using a collision/reaction cell Agilent Technologies 8900x ICP-MS spectrometer. The isotopes 63Cu and 65Cu were monitored in time resolved mode analysis using an integration time of 0.3 sec. 63Cu is the most abundant isotope and therefore this isotope was used to quantify copper by LC-ICP-MS. The general operating parameters are provided in Table 2.

TABLE 2
General operating conditions for speciation

| RF Power | 1550 | W |
|---|---|---|
| RF Matching | 1.80 | V |
| Sample Depth | 9.0 | mm |
| Nebulizer Gas | 1.03 | L/min |
| Option Gas | 0% | |
| Nebulizer Pump | 0.10 | rps |
| S/C Temperature | 2° | C. |

### Data Evaluation

Data were acquired and integrated using MassHunter (version C.01.05 Agilent). Peak area results were exported to Watson LIMS (Version 7.5 SP1, Thermo Scientific) in text file format. Peak areas related to sample analysis were electronically transferred from MassHunter to a Microsoft Excel spreadsheet where the total peak area of each chromatographic constituent of the sample was converted to a percentage of the combined total peak area (all constituents). From the respective percentage and the total copper concentration the ceruloplasmin-bound copper and albumin-bound copper relative concentration results could be determined. All peak integration and subsequent data calculation were subject to independent peer review.

For each peak, the percentage peak area (%) was calculated as follows:

$$( \frac{Peak area of analyte}{( Peak area of all Cu - containing peaks} ) \times 100$$

The relative concentration (ng/mL) for each of the peaks was calculated as follows:

$$( \frac{Total serum Cu concentration ( \frac{ng}{mL} )}{100} ) \times \% Peak Area$$

The copper mass balance (column recovery) was calculated as follows:

$$Copper mass balance = \frac{Mean c opper in LC eluant ( post - column )}{Mean copper in LC eluent ( pre - column )} \times 100$$

### Results

The ceruloplasmin-bound copper levels for each lot of human serum, along with the within-run precision is shown in Table 3 below.

TABLE 3
Mean ceruloplasmin-bound copper levels, total copper concentrations
and within-run precision for six lots of human serum.

| | Mean Calculated | | Within-run |
|---|---|---|---|
| | Cp − Cu Conc | Total Cu Conc | Precision |
| Batch\| | (ng/mL) | (ng/mL) | (RSD %) |
| HMN398561 | 1234 | 1320 | 0.915 |
| HMN398563 | 1151 | 1220 | 0.794 |
| HMN398564 | 828 | 890 | 0.791 |
| HMN398560 | 1138 | 1210 | 0.729 |
| HMN398559 | 1151 | 1220 | 0.795 |
| HMN398558 | 1269 | 1350 | 0.723 |

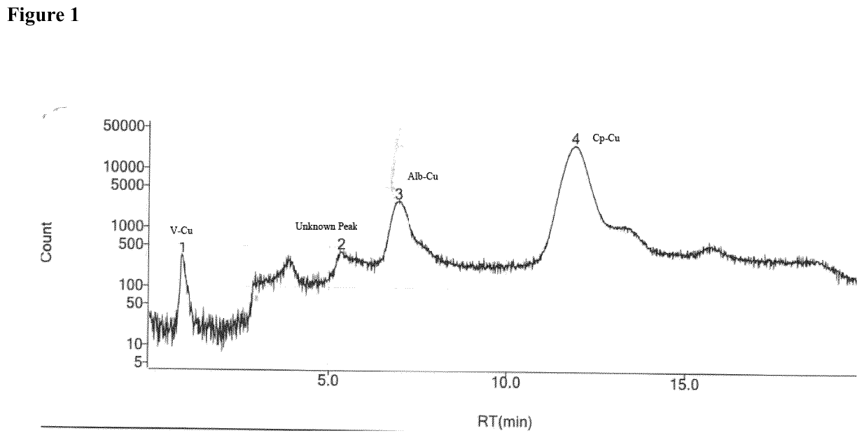

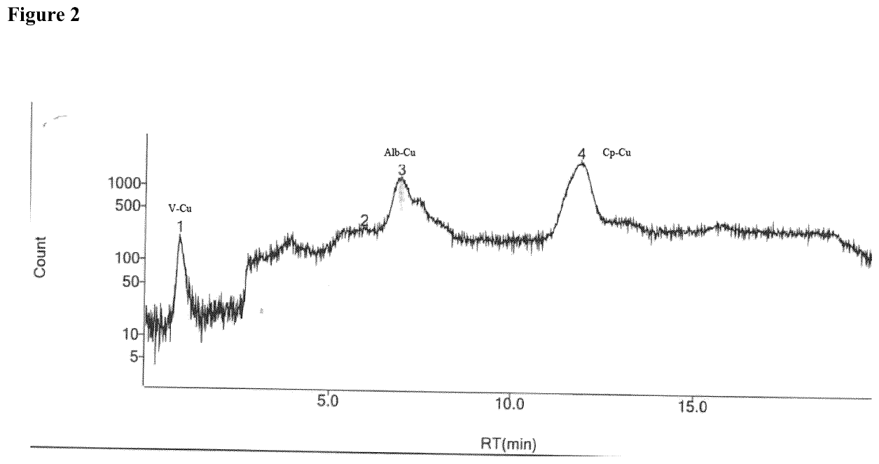

Representative chromatographs from serum samples from a healthy patient and a Wilson's Disease patient, obtained in accordance with the method of Example 2, are shown in FIGS. 1 and 2.

Within-run precision, between-run precision and total precision (assay variability), assessed on three separate occasions were within acceptance limits. The total precision ranged from 0.723 to 0.915%. The influence of endogenous components, demonstrated using six different lots of human serum (matrix variability), had no effect on the quantitation of ceruloplasmin-bound copper. The maximum run size for this assay (assay robustness) was 60 injections per batch.

A copper recovery/mass balance from the LC column performed using serum from six individual subjects demonstrated (recovery 97.7%; RSD % 10.7) speciation of copper using this methodology fully represents all species present.

### Example 3—Treatment of Wilson's Disease Patients with Monitoring by NCC Cu Speciation Assay

A randomized, open-label study was performed to assess the efficacy of trientine tetrahydrochloride treatment for Wilson's Disease with an active standard of-care comparator (D-penicillamine). Efficacy was assessed and treatments were monitored primarily using total copper analysis and a copper speciation assay as outlined in Examples 1 and 2 to determine serum NCC levels.

### Study Overview

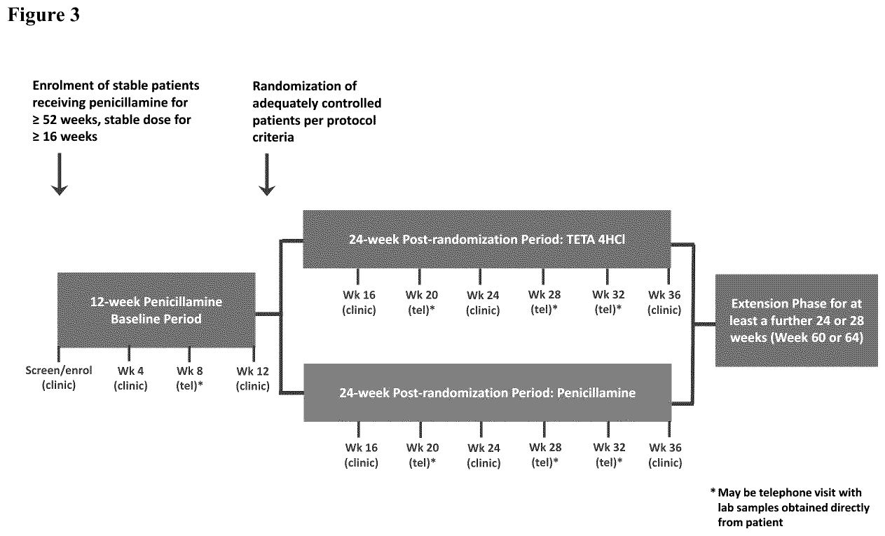

Eligible patients were clinically stable with their standard of-care D penicillamine chelation therapy for at least 1 year. These patients entered a 12-Week Penicillamine Baseline Period (from study visits on Day 1 to Week 12). During this time, all patients continued their current D penicillamine regimen under study conditions. At the end of the Penicillamine Baseline Period, patients who fulfilled the protocol definition of being adequately controlled and tolerating D penicillamine were randomized in a 1:1 ratio to receive either TETA 4HCl or to continue to receive D penicillamine during a 24-Week Post-Randomization Period (from study visits Week 12 to Week 36). The study schematic is shown in FIG. 3 (the extension phase relates to longer term testing for which results are not yet available). A total of 53 patients were randomized into the study and received study treatment (27 in the D penicillamine arm and 26 in the TETA 4HCl arm).

### Patient Population

Key inclusion criteria for participation in the study were:
male or female, aged ≥18 and ≤75 years at time of consent;
patient had a diagnosis of Wilson's disease, as defined by a prior or current Leipzig score of ≥4 the patient's Wilson's disease was clinically stable and was treated with D penicillamine for at least 1 year (52 weeks) prior to the screening/enrollment visit, in particular a stable dose of D penicillamine for at least 4 months (16 weeks) prior to the screening/enrollment visit;
patient was considered suitable to receive therapy with both TETA 4HCl and D penicillamine administered twice a day;
negative central laboratory tests for human immunodeficiency and viral hepatitis.

Adequate control of Wilson's Disease and toleration of D penicillamine therapy was defined by fulfilment of all of the following:
Serum NCC level between ≥25 and ≤150 μg/L
24-hour urinary copper excretion (UCE) between ≥100 and ≤900 μg/24 hours
Alanine transaminase (ALT)<2 times upper limit of normal (ULN)
No other laboratory or clinical findings that would prevent continuation of maintenance therapy

Initial serum NCC levels to determine inclusion criteria for the clinical trial were measured using the standard EDTA ultra-filtration technique. Some patients were found not to meet the initially defined inclusion criterion for controlled NCC levels. However, certain patients were deemed to have controlled Wilson's Disease, despite not meeting this criterion, due to their remaining clinical parameters and 24 hour UCE levels demonstrating effective control of their symptoms. The clinical assessment of these patients was therefore that their disease was controlled. When serum NCC levels were re-tested using the copper speciation assay (as set out in Examples 1 and 2), it was found that the NCC level of these patients was in fact within the inclusion criterion limits, but the previously used EDTA test had produced an inaccurate assessment of their NCC levels.

Any of the following exclusion criteria prevented a patient from participating in the study:
patient was in the “de-coppering” phase of treatment for Wilson's disease;
patient had evidence of uncontrolled liver disease, including but not limited to modified Nazer score of >4, decompensated cirrhosis, acute hemolytic anemia, acute hemolytic anemia, acute hepatitis, hepatic malignancy, evidence of acute liver failure
patient was currently taking medication containing trientine, or was currently receiving zinc therapy, gold therapy, antimalarial therapy, cytotoxic drugs, oxyphenbutazone, or phenylbutazone;
patient had a known intolerance, allergy or sensitivity to D penicillamine (that was uncontrolled) or to TETA 4HCl, including any component of the study medication.

Patients in both treatment arms (D-penicillamine and TETA 4HCl) had similar baseline values for serum NCC, modified Nazer score, serum ceruloplasmin, and AST, although all showed considerable variability across the population, reflecting the diversity of Wilson's disease. Some mean baseline differences were seen between the D penicillamine arm and the TETA 4HCl arm with regard to serum total copper (384.2 and 218.6 μg/L, respectively), and ALT (35.6 and 34.7 U/L, respectively), however considering the inherent variability observed, this was not considered clinically relevant.

### Test Product and Reference Therapy

The test product was Orphalan's orally administered TETA 4HCl film-coated tablets (150 mg trientine base per tablet). The total daily dose administered following randomization (in mg trientine base) was the same total daily dose in mg of D penicillamine administered at the end of the Penicillamine Baseline Period, rounded to the nearest 150 mg of trientine base.

The comparator was the patient's established orally administered standard of-care D penicillamine therapy as prescribed by their treating physician. The total daily dose at enrolment into the Penicillamine Baseline Period was that already established for the patient. At the end of the 12-week Penicillamine Baseline Period, patients were randomized, using the total daily dose administered at the time.

Throughout the study, the total dose was to be divided into 2 equal doses for a twice-a-day (BID) regimen. Patients were instructed to take the study medication with water and swallowed on an empty stomach, separated from the intake of other drugs, food and some drinks. Each administration should have been a) either at least 1 hour before a meal or 2 hours after a meal and b) at least 1 hour apart from any other drug(s), food intake or milk. All patients were instructed to maintain a stable diet throughout the study and avoid food and drinks with high copper content.

### Dose Adjustment

As previously described, the dose selected for each patient was individualized on the basis of their requirement for maintenance chelation therapy. Initially, this was a continuation of their usual maintenance total daily dose of D penicillamine. Thereafter, the dose of study medication depended on the dose for the patient at the end of the preceding phase of the study. However, the total daily dose of study medication (TETA 4HCl or D penicillamine) was altered if the patient was no longer considered to be adequately controlled or tolerating the therapy.

The decision to increase the dose took primarily the following into consideration:
serum NCC>150 μg/L; 24-hour UCE<100 μg/24 hours; ALT level >2 times the upper limit of normal (ULN) or more than twice the patient's baseline value; patient's compliance with therapy and dietary copper intake. The decision to decrease the dose took the following into consideration: serum NCC<25 μg/L; 24-hour UCE>900 μg/24 hours; any safety finding that indicated the patient was not tolerating the study medication at the administered dose (e.g. evidence of copper deficiency); patient's compliance with therapy and dietary copper intake.

The dose of study medication was adjusted to provide the total daily dose anticipated to bring the patient back into being adequately managed and tolerating the therapy. For the purposes of this study, the change in dose was standardized according to the following increments:
If current dose of chelator was ≤1000 mg, increased/decreased total daily dose by 1 tablet/capsule;
If current dose of chelator was >1000 mg, increased/decreased total daily dose by 2 tablets/capsules.

Between the start of Week 12 and the end of Week 36, seven patients in the TETA 4HCl treatment arm received dose modifications in accordance with this protocol.

### Efficacy Evaluation and Statistical Methods

The primary efficacy endpoint was the serum NCC concentration (in μg/L) at Week 36. Serum NCC concentrations were determined using a copper speciation serum NCC assay using the methodology outlined in Examples 1 and 2. The primary efficacy analysis utilized the serum NCC values from all study visits that were analyzed using a restricted maximum likelihood based general linear model for correlated data. The correlation due to repeated measures was modelled by specifying the variance covariance matrix.

Secondary efficacy endpoints were the 24-hour UCE (in μg/24 hours), measured by a central laboratory using a validated assay; clinical stability assessment by the independent Adjudication Committee (blinded to treatment arm post-randomization); and the score on the Clinical Global Impression of Change (CGIC) rating scale (a 7-point scale in which the patient responded to the following statement: “Please rate the change in the overall severity of the patients Wilson's disease compared to the previous study clinic visit.” The responses options were: 1) very much improved, 2) much improved, 3) minimally improved, 4) no change, 5) minimally worse, 6) much worse, or 7) very much worse.”).

These efficacy assessments are in accordance with those generally considered standard for patients with Wilson's Disease (apart from the use of the copper speciation assay for the assessment of serum NCC levels). Of note, the primary efficacy measure was supported by clinically important secondary assessments, including the 24 hour UCE, which is also a common measure for monitoring patients with Wilson's disease.

### Statistical Analysis

The number and the proportion of patients with a serum NCC value that matched the threshold (eg, serum NCC≤50 μg/L) were presented along with the 95% confidence interval of the proportion by visit and treatment arm. The proportions in the 2 arms were compared using Fisher's exact test. The same approach was applied on 24-hour UCE.

Serum NCC and 24-hour UCE within-patient variability was also compared between the 2 treatment arms. The proportions of clinically stable patients were computed and reported with their exact 95% confidence intervals. Fisher's exact test was used to compare the 2 treatment arms for these proportions.

The CGIC rating scale, a 7-point ordinal score, was summarized by visit and treatment arm, and compared between arms using a stratified Cochran-Mantel-Haenszel test. The UWDRS, a continuous measure, was summarized by visit and treatment arm with descriptive statistics for absolute values and the changes from baseline.

For change from baseline in laboratory measurements, “baseline” was defined as the last non-missing value during the Penicillamine Baseline Period, including unscheduled values.

### Results

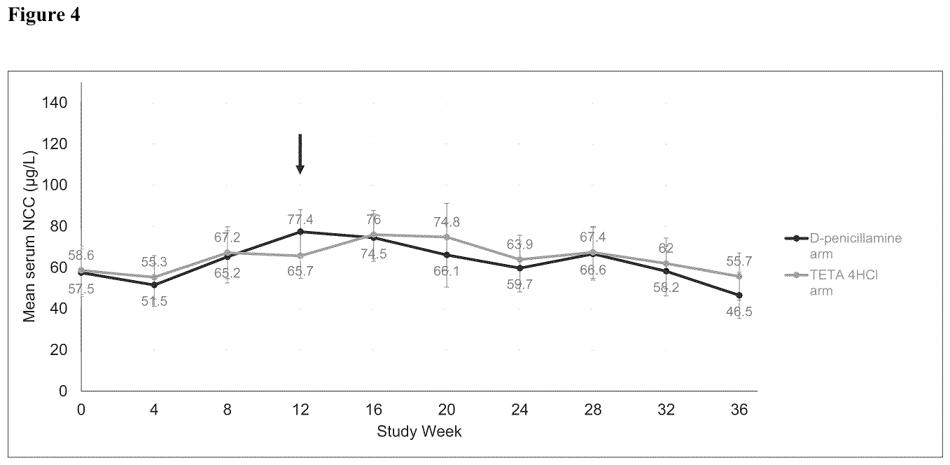

The mean serum NCC up to Week 36 is provided in Table 4 and a summary of the data (adjusted using a statistical model to account for repeat measurements) is displayed in FIG. 4.

TABLE 4
Summary results on serum NCC values, μg/L (ITT population)

| | D-Penicillamine | | TETA 4HCl | |
|---|---|---|---|---|
| | (N = 27) | | (N = 26) | |
| Week and summary | | Change from | | Change from |
| measure | Value | baseline | Value | baseline |

12-Week Penicillamine Baseline Period

| Day 1 | | | NA | | | NA |
|---|---|---|---|---|---|---|
| n | 26 | | | 24 | | |
| Mean (SEM) | 63.9 | (5.31) | | 60.7 | (5.24) | |
| 95% CI of mean | [52.9; 74.8] | | | [49.8; 71.5] | | |
| Median (Q1, Q3) | 61.0 | (42.0,75.0) | | 58.2 | (45.8,69.8) | |
| SD (Min, Max) | 27.09 | (17; 138) | | 25.69 | (26; 149) | |

Week 4

| n | 26 | | NA | 24 | | NA |
|---|---|---|---|---|---|---|
| Mean (SEM) | 59.4 | (4.89) | | 57.6 | (4.43) | |
| 95% CI of mean | [49.3; 69.4] | | | [48.4; 66.7] | | |
| Median (Q1, Q3) | 58.4 | (39.0; 67.7) | | 58.5 | (43.5; 62.0) | |
| SD (Min, Max) | 24.91 | (19; 132) | | 21.69 | (26; 120) | |

Week 8

| n | 27 | | NA | 23 | | NA |
|---|---|---|---|---|---|---|
| Mean (SEM) | 71.9 | (5.89) | | 70.0 | (6.68) | |
| 95% CI of mean | [59.7; 84.0] | | | [56.1; 83.8] | | |
| Median (Q1, Q3) | 69.0 | (50.6; 99.0) | | 61.5 | (46.9; 87.0) | |
| SD (Min, Max) | 30.63 | (27; 140) | | 32.02 | (34; 164) | |

Week 12 (randomization)

| n | 26 | | NA | 25 | | NA |
|---|---|---|---|---|---|---|
| Mean (SEM) | 84.4 | (4.38) | | 68.5 | (6.56) | |
| 95% CI of mean | [75.4; 93.4] | | | [55.0; 82.1] | | |
| Median (Q1, Q3) | 83.0 | (71.3; 99.0) | | 61.1 | (50.8; 87.0) | |
| SD (Min, Max) | 22.35 | (19; 133) | | 32.80 | (21; 182) | |

24-Week Post-Randomization Period

| Week 16 | | | | | | | | |
|---|---|---|---|---|---|---|---|---|
| n | 24 | | 24 | | 26 | | 26 | |
| Mean (SEM) | 82.8 | (5.50) | −2.1 | (3.80) | 78.0 | (6.24) | 9.4 | (3.12) |
| 95% CI of mean | [71.4; 94.1] | | [−9.9; 5.8] | | [65.1; 90.8] | | [2.9; 15.8] | |
| Median (Q1, Q3) | 77.5 | (68.0; 98.5) | 0.0 | (−11.0; 8.3] | 69.6 | (65.0; 94.0) | 9.2 | (−1.1; 17.8) |
| SD (Min, Max) | 26.96 | (27; 128) | 18.63 | (−53; 34] | 31.82 | (34; 179) | 15.90 | (−1.1; 17.8) |

Week 20

| n | 22 | | 22 | | 26 | | 26 | |
|---|---|---|---|---|---|---|---|---|
| Mean (SEM) | 73.3 | (6.01) | −12.3 | (3.36) | 76.8 | (9.05) | 8.2 | (7.23) |
| 95% CI of mean | [60.7; 85.8] | | [−19.3; −5.3] | | [58.2; 95.4] | | [−6.7; 23.1] | |
| Median (Q1, Q3) | 75.5 | (56.1; 85.0) | −11.6 | (−22.0; 3.0) | 60.8 | (47.9; 93.0) | 4.1 | (−8.0; 14.0) |
| SD (Min, Max) | 28.21 | (21; 140) | 15.77 | (−47; 8) | 46.16 | (32; 214) | 38.62 | (21; 214) |

Week 24

| n | 23 | | 23 | | 22 | | 22 | |
|---|---|---|---|---|---|---|---|---|
| Mean (SEM) | 66.3 | (4.37) | −16.9 | (3.61) | 60.3 | (3.78) | −0.4 | (4.13) |
| 95% CI of mean | [57.3; 75.4] | | [−24.4; −9.4] | | [52.4; 68.1] | | [−9.0; 8.2] | |
| Median (Q1, Q3) | 64.0 | (52.0; 81.7) | −20.0 | (−29.0; −8.0) | 57.0 | (47.7; 69.0) | 3.0 | (−12.0; 12.2) |
| SD (Min, Max) | 20.97 | (34; 111) | 17.32 | (−38; 20) | 57.0 | (33; 101) | 3.0 | (−52; 32) |

Week 28

| n | 26 | | 26 | | 23 | | 23 | |
|---|---|---|---|---|---|---|---|---|
| Mean (SEM) | 73.1 | (6.51) | −10.6 | (7.26) | 69.5 | (6.12) | −0.8 | (3.52) |
| 95% CI of mean | [59.7; 86.5] | | [−25.6; 4.3] | | [56.8; 82.2] | | [−8.1; 6.5] | |
| Median (Q1, Q3) | 65.1 | (52.0; 92.8) | −17.5 | (−29.0; −4.6) | 62.4 | (52.4; 82.0) | 0.0 | (−11.0; 9.6) |
| SD (Min, Max) | 33.22 | (39; 200) | 37.00 | (−29.0; −4.6) | 29.34 | (37; 182) | 16.89 | (−34; 40) |

Week 32

| n | 26 | | | | | | | |
|---|---|---|---|---|---|---|---|---|
| Mean (SEM) | 64.6 | (5.06) | −19.1 | (4.88) | 64.0 | (5.95) | −4.6 | (3.75) |
| 95% CI of mean | [54.2; 75.0] | | [−29.2; −9.1] | | [51.7; 76.3] | | [−12.3; 3.1] | |
| Median (Q1, Q3) | 62.0 | (50.0; 79.1) | −21.7 | (−33.0; −7.0) | 60.0 | (48.0; 73.0) | 0.7 | (−19.0; 6.0) |
| SD (Min, Max) | 25.79 | (22; 123) | 24.87 | (−62; 47) | 30.36 | (48.0; 73.0) | 19.10 | (−19.0; 6.0] |

Week 36

| n | 25 | | | | | | | |
|---|---|---|---|---|---|---|---|---|
| Mean (SEM) | 53.4 | (3.66) | −29.7 | (3.20) | 57.6 | (5.83) | −11.0 | (4.27) |
| 95% CI of mean | [45.9; 61.0] | | [−36.3; −23.1] | | [45.6; 69.6] | | [−19.8; −2.2] | |
| Median (Q1, Q3) | 51.0 | (40.0; 70.0) | −34.0 | (−40.0; −22.0) | 51.1 | (45.0; 63.0) | −6.9 | (−24.0; 6.3) |
| SD (Min, Max) | 18.28 | (22; 87) | 16.02 | (−49; 14) | 29.73 | (27; 191) | 21.79 | (−55; 27) |

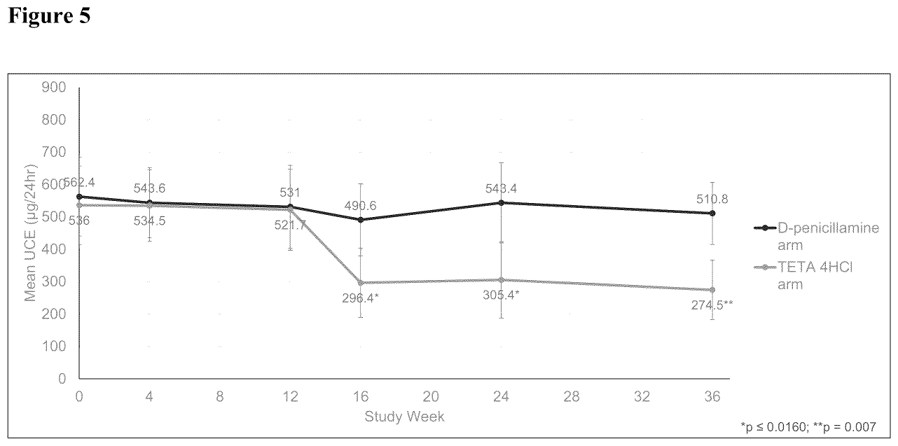

Mean values of 24-hour UCE are provided in Table 5 and FIG. 5 (adjusted as for FIG. 4).

TABLE 5
Summary results of 24-hour UCE values, μg/24 hours (ITT population)

| | D-Penicillamine | | TETA 4HCl | |
|---|---|---|---|---|
| | (N = 27) | | (N = 26) | |
| Week and summary | | Change from | | Change from |
| measure | Value | baseline | Value | baseline |

12-Week Penicillamine Baseline Period

| Day 1 | | | NA | | | NA |
|---|---|---|---|---|---|---|
| n | 27 | | | 26 | | |
| Mean (SEM) | 568.3 | (62.44) | | 550.8 | (49.18) | |
| 95% CI of mean | [439.9; 696.6] | | | [449.6; 652.1] | | |
| Median (Q1, Q3) | 478.8 | (268.2; 823.4) | | 546.6 | (365.4; 720.7) | |
| SD (Min, Max) | 324.47 | (98; 1172) | | 250.78 | (72; 1081) | |

Week 4

| n | 26 | | NA | 23 | | NA |
|---|---|---|---|---|---|---|
| 95% CI of mean | [465.2; 637.9] | | | 410.0; 680.8] | | |
| Mean (SEM) | 551.5 | (41.93) | | 545.4 | (65.28) | |
| Median (Q1, Q3) | 594.6 | (371.2; 689.6) | | 436.1 | (272.0; 785.3) | |
| SD (Min, Max) | 213.81 | (39; 980) | | 313.05 | (127; 1424) | |

Week 12 (randomization)

| n | 25 | | NA | 25 | | NA |
|---|---|---|---|---|---|---|
| Mean (SEM) | 539.1 | (63.05) | | 535.1 | (59.61) | |
| 95% CI of mean | [409.90; 669.2] | | | [412.1; 658.1] | | |
| Median (Q1, Q3) | 496.0 | (371.2; 660.6) | | 590.4 | (268.0; 723.8) | |
| SD (Min, Max) | 315.25 | (18; 1438) | | 298.06 | (43; 1039) | |

24-Week Post-Baseline Period

| Week 16 | | | | | | | | |
|---|---|---|---|---|---|---|---|---|
| n | 24 | | 24 | | 25 | | 25 | |
| Mean (SEM) | 512.1 | (51.84) | −26.8 | (64.44) | 316.4 | (44.82) | −223 | (66.19) |
| 95% CI of mean | [404.8; 619.3] | | [−160; 106.5] | | [223.9; 408.9] | | [−360; −86.9] | |
| Median (Q1, Q3) | 501.4 | (311.7; 636.4) | −60.3 | (−153; 101.4) | 265.6 | (162.4; 387.5) | −221 | (−391; −89.1) |
| SD (Min, Max) | 253.94 | (117; 1179) | 315.68 | (555; 955) | 224.12 | (88; 1208) | 330.94 | (803; 555) |

Week 24

| n | 21 | | 21 | | 22 | | 22 | |
|---|---|---|---|---|---|---|---|---|
| Mean (SEM) | 546.8 | (69.21) | 37.9 | (73.89) | 295.7 | (33.16) | −247 | (69.35) |
| 95% CI of mean | [402.4; 691.1] | | [−116; 192.0] | | [226.8; 364.7] | | [−391; −103] | |
| Median (Q1, Q3) | 495.3 | (297.3; 812.6) | 23.4 | (−163; 200.4) | 257.8 | (173.0; 388.5) | −199 | (−498; −22.5) |
| SD (Min, Max) | 317.15 | (113; 1151) | 338.60 | (611; 706) | 155.52 | (86; 746) | 325.26 | (871; 282) |
| Week 36 | | | | | | | | |
| n | 26 | | 26 | | 26 | | 26 | |
| Mean (SEM) | 519.7 | (41.02) | 4.9 | (54.63) | 289.4 | (40.76) | −258 | (66.23) |
| 95% CI of mean | 453.3; 604.2 | | [−108; 117.4] | | [205.4; 373.3] | | [−394; −121] | |
| Median (Q1, Q3) | 536.9 | (302.1; 661.5) | −4.7 | (−183; 176.4) | 234.8 | (149.8; 353.6) | −231 | (−488; −78.9) |
| SD (Min, Max) | 209.18 | (92; 859) | 278.56 | (579; 553) | 207.84 | (39; 901) | 337.72 | (822; 626) |

As can be seen from Table 4, serum NCC levels (measured using the copper speciation assay described herein) in the D-penicillamine arm and the TETA 4HCl arm were very similar. Determination of serum NCC as measured by a copper speciation assay therefore represents a measurement of Wilson's Disease control that can be used with the same target range independent of the treatment. In contrast, and as shown in Table 5, there were statistically significant differences in 24-hour UCE between treatment arms at all time points analyzed after randomization, with higher levels of copper excretion in the D penicillamine arm. This is attributable to the difference in mechanism of action between TETA 4HCl and D-penicillamine, and means that 24-hour UCE cannot be used with the same target range for all standard Wilson's Disease treatments. It is also considered a less accurate and less reliable measurement for monitoring Wilson's Disease patients.

Table 6 displays results of the assessments using the CGIC rating scale through Week 36. The mean and median remained very close to or at 4 throughout the Penicillamine Baseline Period and 24-week Post-randomization Period in both treatment arms.

TABLE 6
Summary results of the Clinical Global Impression
of Change Scale (ITT population)

| | | | |
|---|---|---|---|
| Week and summary | D-Penicillamine | TETA 4HCl | P |
| measure | (N = 27) | (N = 26) | value* |

12-Week Penicillamine Baseline Period
Week 4D-

| n | 27 | | 26 | | 0.5176 |
|---|---|---|---|---|---|
| Mean (SEM) | 4.1 | (0.09) | 4.0 | (0.04) | |
| Median (Q1, Q3) | 4.0 | (4.0; 4,0) | 4.0 | (4.0; 4,0) | |
| SD (Min, Max) | 0.47 | (3; 6) | 0.20 | (4; 5) | |
| Week 12 | | | | | |
| n | 27 | | 26 | | 0.1094 |
| Mean (SEM) | 3.9 | (0.05) | 4.0 | (0.04) | |
| Median (Q1, Q3) | 4.0 | (4.0; 4,0) | 4.0 | (4.0; 4,0) | |
| SD (Min, Max) | 0.27 | (3; 4) | 0.20 | (4; 5) | |

24-Week Post-Randomization Period
Week 16

| n | 25 | | 26 | | 0.8195 |
|---|---|---|---|---|---|
| Mean (SEM) | 4.1 | (0.06) | 4 | (0.10) | |
| Median (Q1, Q3) | 4.0 | (4.0; 4,0) | 4.0 | (4.0; 4,0) | |
| SD (Min, Max) | 0.28 | (4; 5) | 0.49 | (3; 5) | |

Week 24

| n | 26 | | 25 | | 0.9570 |
|---|---|---|---|---|---|
| Mean (SEM) | 4.0 | (0.04) | 4.0 | (0.11) | |
| Median (Q1, Q3) | 4.0 | (4.0; 4,0) | 4.0 | (4.0; 4,0) | |
| SD (Min, Max) | 0.2 | (3; 4) | 0.54 | (3; 5) | |

Week 36

| n | 26 | | 26 | | 0.4581 |
|---|---|---|---|---|---|
| Mean (SEM) | 4.0 | (0.0) | 3.9 | (0.14) | |
| Median (Q1, Q3) | 4.0 | (4.0; 4,0) | 4.0 | (4.0; 4,0) | |
| SD (Min, Max) | 0.00 | (4; 4) | 0.49 | (3; 5) | |

For the Unified Wilson's Disease Rating Scale, there were no notable changes in mean scores within each treatment arm during the Penicillamine Baseline Period or in the Post-Randomization Period and no notable difference between treatment arms was observed.

### Summary

The results show that subjects undergoing treatment for Wilson's Disease by administration of either trientine tetrahydrochloride or D-penicillamine can be effectively monitored by assessing serum NCC levels using the reliable and accurate copper speciation assay described in Examples 1 and 2. The serum NCC levels as measured using the copper speciation assay are shown to be consistent with known monitoring techniques (such as 24 hour UCE) and can therefore be used alone as a monitoring technique.

The results also show that Wilson's Disease can be effectively controlled by monitoring the serum NCC level of patients with Wilson's Disease and adapting the treatment dosage to control serum NCC levels to be within a specific range, as measured using the copper speciation assay. The results further show that serum NCC levels of the subjects can be appropriately adjusted by modifying the treatment dose to bring the serum NCC level into the desired range. This provides a new approach for optimized treatment of Wilson's Disease, using the copper speciation assay.

### Example 4—Serum NCC Range of Healthy Volunteers

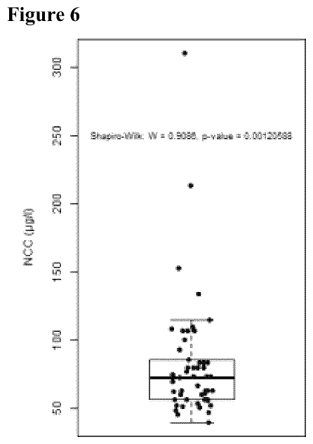

Serum samples were obtained from 50 healthy volunteers. 25 females and males with a mean (range) age of 40 (21-62) and 40 (20-78) years of whom 60% were Hispanic, participated. Results of the copper speciation are shown in Table 7 and displayed as a Shapiro-Wilk plot in FIG. 6. As the hypothesis of normality was rejected, the reference interval was calculated using a non-parametric method, i.e. the reference interval limits are the 2.5th and 97.5th percentiles. This method makes no assumption about the probability distribution of the observed values. Outliers were consistently present by subgroup (gender, age, race). Two outlying observations (NCC=310 and 213 μg/l) were identified using Cook's distance and removed. The confidence interval for the lower and upper limits were calculated using bootstrapping (with 5000 bootstrap samples). The reference interval limits are 41.2 (90% CI 34.5; 42.4) and 148.7 (90% CI 144.5; 184).

The serum NCC levels were measured using the methods described in Examples 1 and 2. The results are shown in Table 7.

TABLE 7
Serum NCC levels of healthy volunteers

| | | Cp − Cu | Alb-Cu | |
|---|---|---|---|---|
| | Total Copper | relative | relative | |
| | concentration | abundance | abundance | NCC |
| | (ng/mL) | (ng/mL) | (ng/mL) | (ng/mL) |
| Mean | 1163 | 1081 | 70.0 | 82.2 |
| Median | 1145 | 1065 | 58.2 | 72.8 |
| n | 50 | 50 | 50 | 50 |
| SD | 279 | 288 | 43.8 | 45.0 |
| % RSD | 24.0 | 26.6 | 62.6 | 54.7 |
| Min | 724 | 414 | 33.3 | 40.0 |
| Max | 1960 | 1870 | 295 | 310 |

Conclusion: A reference interval was established for the NCC-CuSp assay as 41.2-148.7, (i.e. 40 to 150 ng/mL) based on the results of 50 healthy volunteers.

### Example 5

Patients enrolled in the clinical trial of Example 3 had blood samples taken for NCC evaluation by both methods at screening and 9 visits every 4 weeks thereafter from 53 WD patients. Paired data were compared using Bland Altman (BA) analysis. The standard deviation (SD) of this difference was calculated based on a mixed model, taking into account repeated measures for each subject.

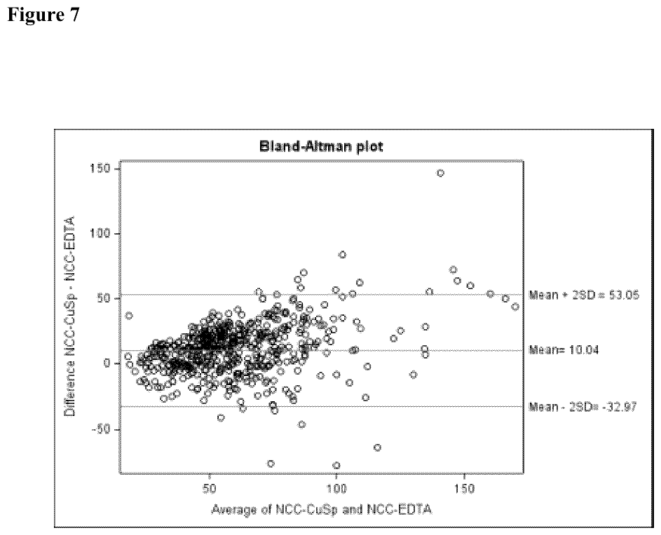

Results: 511 paired samples were available from 73 patients. The BA plot (FIG. 7) shows a mean difference of 10.04, with (2SD) limits of agreement (−32.97, 53.05) microgram/L.

Conclusion: NCC EDTA is both inaccurate, underestimating NCC CuSp (copper speciation) by an average 10 microgram/L and imprecise with a greater tendency to underestimate the NCC-CuSp across the range of values measured.

### Example 6

The serum NCC levels of patients with Wilson's Disease (enrolled in the clinical trial of Example 3) and healthy volunteers were measured using the methods described in Examples 1 and 2. Further statistical analysis was carried out. For the healthy volunteer data, no categorisation was performed and the results are based on a single specimen from each participant. For the Wilson's Disease patient group, the analysis was performed on data taken from patients post-randomisation receiving either D-penicillamine or TETA 4HCl over a 48 week period (sampled at 4, 8, 12, 16, 20, 24 and 48 weeks), who were considered to be stable and well-controlled, i.e. they had stable disease. Stable disease is defined as ALT<82 or <1.5× upper limit of normal. Table 8 provides the resulting descriptive statistics.

TABLE 8
Summary of NCC ranges at 95% reference interval

| | | Wilson's Disease patient |
|---|---|---|
| | Healthy patient group | group NCC, ng/mL |
| | NCC, ng/mL | (Example 3) |
| Mean | 116 | 67 |
| Median | 86 | 61 |
| n | 75 | 52 |
| Q1 | 63 | 46 |
| Q3 | 171 | 80 |
| RI lower limit | 45 | 25 |
| (rounded) | | |
| RI upper limit | 215 | 130 |
| (rounded) | | |
| SD | 62 | 32 |
| Minimum | 40 | 16 |
| Maximum | 314 | 214 |

What is claimed is:

1. A copper (II) chelator for use in a method of treating Wilson's Disease in a subject, wherein the method comprises
measuring a serum non-ceruloplasmin bound copper level of the subject using a copper speciation assay; and
controlling the subject's serum non-ceruloplasmin bound copper level to be in the range of from 25 to 130 ng/mL.

2. A copper (II) chelator for use in a method of monitoring a subject undergoing treatment for Wilson's Disease by administration of the copper (II) chelator, wherein the method comprises
measuring a serum non-ceruloplasmin bound copper level of the subject using a copper speciation assay; and
controlling the subject's serum non-ceruloplasmin bound copper level to be in the range of from 25 to 130 ng/mL.

3. A copper (II) chelator for use according to claim 1 or claim 2, wherein the range is from 40 to 80 ng/mL.

4. A copper (II) chelator for use according to any one of the preceding claims, wherein said measuring step is repeated one or more times during the subject's treatment, such that the subject's serum non-ceruloplasmin levels are monitored during their treatment.

5. A copper (II) chelator for use according to claim 3 or claim 4, which method comprises measuring or monitoring the subject's serum non-ceruloplasmin bound copper level and, in the case that the subject's serum non-ceruloplasmin bound copper level falls outside the range of from 40 to 80 ng/mL, adjusting the dose of copper (II) chelator, and optionally maintaining the adjusted dose until the subject's serum non-ceruloplasmin bound copper level is once again within the range of from 40 to 80 ng/mL.

6. A copper (II) chelator for use according to claim 5, wherein said adjustment comprises increasing the dose of the copper (II) chelator if the subject's serum non-ceruloplasmin bound copper level falls above 80 ng/mL, preferably increasing the dose by 150 to 300 mg/day of active chelator, or reducing the dose of the copper (II) chelator if the subject's serum non-ceruloplasmin bound copper level falls below 40 ng/mL preferably reducing the dose by 150 to 300 mg/day of active chelator.

7. A copper (II) chelator for use in a method of treating Wilson's Disease in a subject, the method comprising:
a) administering to the subject a therapeutically effective dose of the copper (II) chelator;
b) measuring a serum non-ceruloplasmin bound copper level of the subject after administration of the dose of the copper (II) chelator using a copper speciation assay; and
c) determining whether the serum non-ceruloplasmin bound copper level of the subject after administration of the dose of the copper (II) chelator is within a range of from 25 to 130 ng/mL, preferably from 40 to 80 ng/mL.

8. A copper (II) chelator for use according to any one of claims 1 to 7, wherein the copper (II) chelator is trientine tetrahydrochloride or D-penicillamine, preferably trientine tetrahydrochloride.

9. A copper (II) chelator for use according to claim 7, the method comprising:
a) administering to the subject at least a first and a further therapeutically effective dose of the copper (II) chelator;
b) measuring a serum non-ceruloplasmin bound copper level of the subject after administration of the first dose of the copper (II) chelator using a copper speciation assay;
c) determining whether the serum non-ceruloplasmin bound copper level of the subject after administration of the first dose of the copper (II) chelator is within a range of from 40 to 80 ng/mL; and either
d) if the serum non-ceruloplasmin bound copper level of the subject after administration of the first dose of the copper (II) chelator is not within a range of from 40 to 80 ng/mL, modifying the further dose of the copper (II) chelator compared to the first dose; or
e) if the serum non-ceruloplasmin bound copper level of the subject after administration of the first dose of the copper (II) chelator is within a range of from 40 to 80 ng/mL, keeping the further dose the same as the first dose.

10. A copper (II) chelator for use according to claim 9, wherein the copper (II) chelator is trientine tetrahydrochloride or D-penicillamine, preferably trientine tetrahydrochloride.

11. A copper (II) chelator for use according to claim 9 or claim 10, wherein
if the serum non-ceruloplasmin bound copper level of the subject after administration of the first dose of the copper (II) chelator is below 40 ng/mL, the further dose is reduced compared to the first dose, or
if the serum non-ceruloplasmin bound copper level of the subject after administration of the first dose of the copper (II) chelator is above 80 ng/mL, the further dose is increased compared to the first dose.

12. A copper (II) chelator for use according to claim 11, wherein
if the further dose is reduced, the further dose is reduced by 150 to 300 mg/day of active chelator compared to the first dose, or
if the further dose is increased, the further dose is increased by 150 to 300 mg/day of active chelator compared to the first dose.

13. A method of assessing a subject who is susceptible to Wilson's Disease, the method comprising:
a) measuring a serum non-ceruloplasmin bound copper level of the subject using a copper speciation assay; and
b) determining whether the serum non-ceruloplasmin bound copper level of the subject is above 150 ng/mL.

14. A copper (II) chelator for use according to any one of claims 1 to 12, or a method according to claim 13, wherein the serum non-ceruloplasmin bound copper level is measured using the following steps:
i) determining the total serum copper level;
ii) determining the serum ceruloplasmin-derived copper level using a copper speciation assay, preferably using a chromatographic method; and
iii) calculating the serum non-ceruloplasmin bound copper level by subtracting the value obtained in step ii) from the value obtained in step i).

15. A copper (II) chelator for use or a method according to claim 14, wherein the total serum copper level is determined using ICP-MS and the serum ceruloplasmin-derived copper level is determined using an anion exchange chromatographic method, preferably LC-ICP-MS.
